SEQUENCE LISTING (22 sequences)

SEQ ID NO: 1 - protein, 289 aa, synthetic construct
METDTLLLWVLLLWVPGSTGDAAQPARRAKRSLEEKKGNYVVTDHAAAGSPGPPGPPGPPGPPGPPGPPGPPGPPGPPGPPGPSSGGTGGDEDPQIAAHVVSEANSNAASVLQWAKKGYYTMKSNLVMLENGKQLTVKREGLYYVYTQVTFCSNREPSSQRPFIVGLWLKPSSGSERILLKAANTHSSSQLCEQQSVHLGGVFELQAGASVFVNVTEASQVIHRVGFSSFGLLKLGSLVPRGSPGGSWSHPQFEKRRGGPEQKLISEEDLNSAVDGSSHHHHHHSRGLE

SEQ ID NO: 2 - DNA, 870 bp, synthetic construct
  source 1..870: mol_type = other DNA
atggagacagacacactcctgctatgggtactgctgctctgggttccaggttccactggtgacgcggcccagccggccaggcgcgccaaacgatcactggaggagaagaagggcaactacgtggtgaccgaccacgcggccgctggcagccctgggccacctggtcccccaggtcctccaggacccccagggcccccaggcccccccgggccgcctggacccccagggccaccaggccccccaggcccttcctctggcggaaccggtggcgacgaagatcctcaaatcgccgcacatgtcgtgagcgaggcaaacagtaacgcagcaagtgttctgcaatgggctaaaaaagggtactacactatgaaatccaacttggtaatgctcgaaaacggcaaacagctcaccgtcaaacgagaagggctctattatgtttacacacaggtaactttttgttcaaatagggaaccttctagtcaacgtcctttcattgtgggactgtggctcaagcctagttcaggcagcgagagaatcttgttgaaagcagctaacactcattcctcttcacaattgtgcgagcaacagtcagtacacttgggaggagtatttgagttgcaagctggtgcctctgttttcgttaatgtgaccgaagccagccaggtaatccatcgagtaggtttttcttcatttgggcttctcaagctcggatccctggtgccgcgcggcagccctggaggctcctggagccacccgcagttcgaaaagcgtcgaggagggccagaacaaaaactcatctcagaagaggatctgaatagcgccgtcgacggcagcagccatcatcaccatcaccatagccgcggactcgagtaa

SEQ ID NO: 3 - protein, 396 aa, synthetic construct
METDTLLLWVLLLWVPGSTGDAAQPARRAKRSLEEKKGNYVVTDHASQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGAAAGSPGPPGPPGPPGPPGPPGPPGPPGPPGPPGPPGPSSGGTGGDEDPQIAAHVVSEANSNAASVLQWAKKGYYTMKSNLVMLENGKQLTVKREGLYYVYTQVTFCSNREPSSQRPFIVGLWLKPSSGSERILLKAANTHSSSQLCEQQSVHLGGVFELQAGASVFVNVTEASQVIHRVGFSSFGLLKLGSLVPRGSPGGSWSHPQFEKRRGGPEQKLISEEDLNSAVDGSSHHHHHHSRGLE

SEQ ID NO: 4 - DNA, 1191 bp, synthetic construct
  source 1..1191: mol_type = other DNA
atggagacagacacactcctgctatgggtactgctgctctgggttccaggttccactggtgacgcggcccagccggccaggcgcgccaaacgatcactggaggagaagaagggcaactacgtggtgaccgaccacgctagccagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtgcggccgctggcagccctgggccacctggtcccccaggtcctccaggacccccagggcccccaggcccccccgggccgcctggacccccagggccaccaggccccccaggcccttcctctggcggaaccggtggcgacgaagatcctcaaatcgccgcacatgtcgtgagcgaggcaaacagtaacgcagcaagtgttctgcaatgggctaaaaaagggtactacactatgaaatccaacttggtaatgctcgaaaacggcaaacagctcaccgtcaaacgagaagggctctattatgtttacacacaggtaactttttgttcaaatagggaaccttctagtcaacgtcctttcattgtgggactgtggctcaagcctagttcaggcagcgagagaatcttgttgaaagcagctaacactcattcctcttcacaattgtgcgagcaacagtcagtacacttgggaggagtatttgagttgcaagctggtgcctctgttttcgttaatgtgaccgaagccagccaggtaatccatcgagtaggtttttcttcatttgggcttctcaagctcggatccctggtgccgcgcggcagccctggaggctcctggagccacccgcagttcgaaaagcgtcgaggagggccagaacaaaaactcatctcagaagaggatctgaatagcgccgtcgacggcagcagccatcatcaccatcaccatagccgcggactcgagtaa

SEQ ID NO: 5 - protein, 392 aa, synthetic construct
METDTLLLWVLLLWVPGSTGDAAQPARRAKRSDYWGQGTELASQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGAAAGSPGPPGPPGPPGPPGPPGPPGPPGPPGPPGPPGPSSGGTGGDEDPQIAAHVVSEANSNAASVLQWAKKGYYTMKSNLVMLENGKQLTVKREGLYYVYTQVTFCSNREPSSQRPFIVGLWLKPSSGSERILLKAANTHSSSQLCEQQSVHLGGVFELQAGASVFVNVTEASQVIHRVGFSSFGLLKLGSLVPRGSPGGSWSHPQFEKRRGGPEQKLISEEDLNSAVDGSSHHHHHHSRGLE

SEQ ID NO: 6 - DNA, 1179 bp, synthetic construct
  source 1..1179: mol_type = other DNA
atggagacagacacactcctgctatgggtactgctgctctgggttccaggttccactggtgacgcggcccagccggccaggcgcgccaaacgatcagactactggggccaaggcacagaactagctagccagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtgcggccgctggcagccctgggccacctggtcccccaggtcctccaggacccccagggcccccaggcccccccgggccgcctggacccccagggccaccaggccccccaggcccttcctctggcggaaccggtggcgacgaagatcctcaaatcgccgcacatgtcgtgagcgaggcaaacagtaacgcagcaagtgttctgcaatgggctaaaaaagggtactacactatgaaatccaacttggtaatgctcgaaaacggcaaacagctcaccgtcaaacgagaagggctctattatgtttacacacaggtaactttttgttcaaatagggaaccttctagtcaacgtcctttcattgtgggactgtggctcaagcctagttcaggcagcgagagaatcttgttgaaagcagctaacactcattcctcttcacaattgtgcgagcaacagtcagtacacttgggaggagtatttgagttgcaagctggtgcctctgttttcgttaatgtgaccgaagccagccaggtaatccatcgagtaggtttttcttcatttgggcttctcaagctcggatccctggtgccgcgcggcagccctggaggctcctggagccacccgcagttcgaaaagcgtcgaggagggccagaacaaaaactcatctcagaagaggatctgaatagcgccgtcgacggcagcagccatcatcaccatcaccatagccgcggactcgagtaa

SEQ ID NO: 7 - protein, 391 aa, synthetic construct
METDTLLLWVLLLWVPGSTGDAAQPARRAKRSSIINFEKLASQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGAAAGSPGPPGPPGPPGPPGPPGPPGPPGPPGPPGPPGPSSGGTGGDEDPQIAAHVVSEANSNAASVLQWAKKGYYTMKSNLVMLENGKQLTVKREGLYYVYTQVTFCSNREPSSQRPFIVGLWLKPSSGSERILLKAANTHSSSQLCEQQSVHLGGVFELQAGASVFVNVTEASQVIHRVGFSSFGLLKLGSLVPRGSPGGSWSHPQFEKRRGGPEQKLISEEDLNSAVDGSSHHHHHHSRGLE

SEQ ID NO: 8 - DNA, 1176 bp, synthetic construct
  source 1..1176: mol_type = other DNA
atggagacagacacactcctgctatgggtactgctgctctgggttccaggttccactggtgacgcggcccagccggccaggcgcgccaaacgatcaagtatcatcaactttgaaaagctggctagccagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtgcggccgctggcagccctgggccacctggtcccccaggtcctccaggacccccagggcccccaggcccccccgggccgcctggacccccagggccaccaggccccccaggcccttcctctggcggaaccggtggcgacgaagatcctcaaatcgccgcacatgtcgtgagcgaggcaaacagtaacgcagcaagtgttctgcaatgggctaaaaaagggtactacactatgaaatccaacttggtaatgctcgaaaacggcaaacagctcaccgtcaaacgagaagggctctattatgtttacacacaggtaactttttgttcaaatagggaaccttctagtcaacgtcctttcattgtgggactgtggctcaagcctagttcaggcagcgagagaatcttgttgaaagcagctaacactcattcctcttcacaattgtgcgagcaacagtcagtacacttgggaggagtatttgagttgcaagctggtgcctctgttttcgttaatgtgaccgaagccagccaggtaatccatcgagtaggtttttcttcatttgggcttctcaagctcggatccctggtgccgcgcggcagccctggaggctcctggagccacccgcagttcgaaaagcgtcgaggagggccagaacaaaaactcatctcagaagaggatctgaatagcgccgtcgacggcagcagccatcatcaccatcaccatagccgcggactcgagtaa

SEQ ID NO: 9 - protein, 396 aa, synthetic construct
METDTLLLWVLLLWVPGSTGDAAQPARRAGDEDPQIAAHVVSEANSNAASVLQWAKKGYYTMKSNLVMLENGKQLTVKREGLYYVYTQVTFCSNREPSSQRPFIVGLWLKPSSGSERILLKAANTHSSSQLCEQQSVHLGGVFELQAGASVFVNVTEASQVIHRVGFSSFGLLKLRTGPSSGGASQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGAAAGSPGPPGPPGPPGPPGPPGPPGPPGPPGPPGPPGPSSGGTGSIINFEKLGSLVPRGSPGGSWSHPQFEKRRGGPEQKLISEEDLNSAVDGSSHHHHHHSRGLE

SEQ ID NO: 10 - DNA, 1191 bp, synthetic construct
  source 1..1191: mol_type = other DNA
atggagacagacacactcctgctatgggtactgctgctctgggttccaggttccactggtgacgcggcccagccggccaggcgcgccggcgacgaagatcctcaaatcgccgcacatgtcgtgagcgaggcaaacagtaacgcagcaagtgttctgcaatgggctaaaaaagggtactacactatgaaatccaacttggtaatgctcgaaaacggcaaacagctcaccgtcaaacgagaagggctctattatgtttacacacaggtaactttttgttcaaatagggaaccttctagtcaacgtcctttcattgtgggactgtggctcaagcctagttcaggcagcgagagaatcttgttgaaagcagctaacactcattcctcttcacaattgtgcgagcaacagtcagtacacttgggaggagtatttgagttgcaagctggtgcctctgttttcgttaatgtgaccgaagccagccaggtaatccatcgagtaggtttttcttcatttgggcttctcaagctccgtacgggcccttcctctggcggagctagccagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtgcggccgctggcagccctgggccacctggtcccccaggtcctccaggacccccagggcccccaggcccccccgggccgcctggacccccagggccaccaggccccccaggcccttcctctggcggaaccggtagtatcatcaactttgaaaagctgggatccctggtgccgcgcggcagccctggaggctcctggagccacccgcagttcgaaaagcgtcgaggagggccagaacaaaaactcatctcagaagaggatctgaatagcgccgtcgacggcagcagccatcatcaccatcaccatagccgcggactcgagtaa

SEQ ID NO: 11 - protein, 381 aa, synthetic construct
METDTLLLWVLLLWVPGSTGDAAQPARRAKRSQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGAAAGSPGPPGPPGPPGPPGPPGPPGPPGPPGPPGPPGPSSGGTGGDEDPQIAAHVVSEANSNAASVLQWAKKGYYTMKSNLVMLENGKQLTVKREGLYYVYTQVTFCSNREPSSQRPFIVGLWLKPSSGSERILLKAANTHSSSQLCEQQSVHLGGVFELQAGASVFVNVTEASQVIHRVGFSSFGLLKLGSLVPRGSPGGSWSHPQFEKRRGGPEQKLISEEDLNSAVDGSSHHHHHHSRGLE

SEQ ID NO: 12 - DNA, 1146 bp, synthetic construct
  source 1..1146: mol_type = other DNA
atggagacagacacactcctgctatgggtactgctgctctgggttccaggttccactggtgacgcggcccagccggccaggcgcgccaaacgatcacagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtgcggccgctggcagccctgggccacctggtcccccaggtcctccaggacccccagggcccccaggcccccccgggccgcctggacccccagggccaccaggccccccaggcccttcctctggcggaaccggtggcgacgaagatcctcaaatcgccgcacatgtcgtgagcgaggcaaacagtaacgcagcaagtgttctgcaatgggctaaaaaagggtactacactatgaaatccaacttggtaatgctcgaaaacggcaaacagctcaccgtcaaacgagaagggctctattatgtttacacacaggtaactttttgttcaaatagggaaccttctagtcaacgtcctttcattgtgggactgtggctcaagcctagttcaggcagcgagagaatcttgttgaaagcagctaacactcattcctcttcacaattgtgcgagcaacagtcagtacacttgggaggagtatttgagttgcaagctggtgcctctgttttcgttaatgtgaccgaagccagccaggtaatccatcgagtaggtttttcttcatttgggcttctcaagctcggatccctggtgccgcgcggcagccctggaggctcctggagccacccgcagttcgaaaagcgtcgaggagggccagaacaaaaactcatctcagaagaggatctgaatagcgccgtcgacggcagcagccatcatcaccatcaccatagccgcggactcgagtaa

SEQ ID NO: 13 - protein, 397 aa, synthetic construct
METDTLLLWVLLLWVPGSTGDAAQPARRAKRSQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGAAAGSPGPPGPPGPPGPPGPPGPPGPPGPPGPPGPPGPSSGGTGAQLVAQNVLLIDGPLSWYSDPGLAGVSLTGGLSYKEDTKELVVAKAGVYYVFFQLELRRVVAGEGSGSVSLALHLQPLRSAAGAAALALTVDLPPASSEARNSAFGFQGRLLHLSAGQRLGVHLHTEARARHAWQLTQGATVLGLFRVTPEIPAGLPSPRSEGSLVPRGSPGGSWSHPQFEKRRGGPEQKLISEEDLNSAVDGSSHHHHHHSRGLE

SEQ ID NO: 14 - DNA, 1194 bp, synthetic construct
  source 1..1194: mol_type = other DNA
atggagacagacacactcctgctatgggtactgctgctctgggttccaggttccactggtgacgcggcccagccggccaggcgcgccaaacgatcacagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtgcggccgctggcagccctgggccacctggtcccccaggtcctccaggacccccagggcccccaggcccccccgggccgcctggacccccagggccaccaggccccccaggcccttcctctggcggaaccggtgcccagctggtggctcagaacgtcctgctgatcgacggaccactgagctggtactccgatcccggactggctggggtgtctctgactggcggactgagttataaggaggataccaaagaactggtggtcgcaaaggccggggtgtactatgtcttctttcagctggagctgaggagagtggtcgctggggaaggttctggcagtgtgtcactggcactgcacctgcagccactgaggtctgcagctggagcagcagctctggcactgacagtggacctgccacctgcaagctccgaggcacgaaattccgctttcggttttcagggcaggctgctgcacctgagcgctggacagagactgggggtgcacctgcataccgaagctcgggcacgccatgcatggcagctgacccagggagccacagtcctggggctgtttagggtcactcccgagattcccgccggtctgccttcaccacggtctgagggatccctggtgccgcgcggcagccctggaggctcctggagccacccgcagttcgaaaagcgtcgaggagggccagaacaaaaactcatctcagaagaggatctgaatagcgccgtcgacggcagcagccatcatcaccatcaccatagccgcggactcgagtaa

SEQ ID NO: 15 - protein, 20 aa, Homo sapiens
METDTLLLWVLLLWVPGSTG

SEQ ID NO: 16 - protein, 4 aa, Homo sapiens
RAKR

SEQ ID NO: 17 - protein, 13 aa, Homo sapiens
LEEKKGNYVVTDH

SEQ ID NO: 18 - protein, 9 aa, Mus musculus
DYWGQGTEL

SEQ ID NO: 19 - protein, 8 aa, Gallus gallus
SIINFEKL

SEQ ID NO: 20 - protein, 105 aa, Homo sapiens
QPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 21 - protein, 30 aa, synthetic construct
GPPGPPGPPGPPGPPGPPGPPGPPGPPGPP

SEQ ID NO: 22 - protein, 146 aa, Mus musculus
GDEDPQIAAHVVSEANSNAASVLQWAKKGYYTMKSNLVMLENGKQLTVKREGLYYVYTQVTFCSNREPSSQRPFIVGLWLKPSSGSERILLKAANTHSSSQLCEQQSVHLGGVFELQAGASVFVNVTEASQVIHRVGFSSFGLLKL

(19) United States
(12) Patent Application Publication
(10) Pub. No.: US 2025/0333474 A1
(43) Pub. Date: Oct. 30, 2025

(54) SOLUBLE MULTIMERIC FUSION PROTEINS AND METHODS OF TREATMENT USING THE FUSION PROTEINS

(71) Applicant: INDUSTRIAL TECHNOLOGY RESEARCH INSTITUTE, Hsinchu (TW)

(72) Inventors: Min-Yuan CHOU, Taipei City (TW); Chuan-Chuan HUANG, Hsinchu City (TW); Wei-Chun CHIU, Hsinchu City (TW); Ming-Hua YANG, Zhubei City (TW); Mei-Wei LIN, Zhubei City (TW); Hsiu-Chuan LI, Hsinchu County (TW)

(73) Assignee: INDUSTRIAL TECHNOLOGY RESEARCH INSTITUTE, Hsinchu (TW)

(21) Appl. No.: 19/186,744

(22) Filed: Apr. 23, 2025

Related U.S. Application Data
(60) Provisional application No. 63/638,609, filed on Apr. 25, 2024.

(51) Int. Cl. C07K 14/705; A61K 38/00; A61K 40/19; A61K 40/24; A61K 40/34; A61K 40/42; A61P 35/00; C07K 14/71; C07K 14/77; C07K 14/78

(52) U.S. Cl. CPC C07K 14/70575; A61K 40/19; A61K 40/24; A61K 40/34; A61K 40/4204; A61P 35/00; C07K 14/71; C07K 14/77; C07K 14/78; A61K 38/00; C07K 2319/00

(57) ABSTRACT

Disclosed is a multimeric fusion protein and a method for producing the multimeric fusion protein. The method includes expressing in an mammalian cell a nucleic acid coding for an amino acid sequence comprising, in an N-terminal to C-terminal direction, a signal peptide, (optionally) an antigenic peptide, a CH3 domain of human IgG₁, a (G-P-P)₁₀ collagen-like domain, and a TNF ligand superfamily extracellular domain, the extracellular domain being devoid of a coiled-coil trimerization motif, and allowing the polypeptides expressed in the mammalian cell from the nucleic acid to at least one of trimerize and hexamerize into one or more multimeric fusion proteins.

### CROSS-REFERENCE TO RELATED APPLICATIONS

This application claims priority under 35 U.S.C. § 119(e) to U.S. Provisional Application No. 63/638,609, filed on Apr. 25, 2024, the contents of which are hereby expressly incorporated by reference in their entirety.

### REFERENCE TO ELECTRONIC SEQUENCE LISTING

The application contains a Sequence Listing which has been submitted electronically in .XML format and is hereby incorporated by reference in its entirety. Said .XML copy, created on Apr. 22, 2025, is named “2668-0140PUS1.xml” and is 32,225 bytes in size. The sequence listing contained in this .XML file is part of the specification and is hereby incorporated by reference herein in its entirety.

### BACKGROUND

Antigen presenting cells, such as dendritic cells, macrophages, B cells, are immune cells that play an important role in initiating and coordinating an adaptive immune response by processing and presenting antigens to T cells. Antigen presenting cells engulf and break down antigens into smaller peptides, process the peptides and bind them to major histocompatibility complex (MHC) molecules on their surface, and migrate to lymph nodes, where they encounter T cells. If a T cell recognizes the antigen-MHC complex, it becomes activated and initiates an immune response.

Dendritic cells are the most potent antigen presenting cells and play a critical role in innate and adaptive immunity. Dendritic cells secrete a wide array of proteins, including cytokines, chemokines, and other molecules, to orchestrate immune responses, including activating T cells and modulating inflammation. One major challenge in developing dendritic cell-targeted therapies is the effective delivery of antigen to dendritic cells to facilitate the activation of antigen-specific CD8+ cytotoxic T cells.

For example, it has been difficult to activate dendritic cells because they require two non-self antigens, i.e., one of the MHC class I and one of the MHC class II. MHC I is involved in presenting endogenous antigens to CD8+ T cells and is expressed constitutively, while MHC II is involved in presenting exogenous antigens to CD4+ T cells and is regulated and upregulated during dendritic cell maturation. Peptide-MHC I complexes on dendritic cells are recognized by CD8+ T cells, leading to their activation and the elimination of infected or cancerous cells. MHC II molecules present peptides derived from proteins taken up by the cell from exogenous antigens. Peptide-MHC II complexes on dendritic cells are recognized by CD4+ T cells, leading to their activation and the development of helper T cell responses.

CD40 ligand (CD40L, also known as CD154) is a member of the tumor necrosis factor (TNF) superfamily and is primarily expressed on activated CD4+ T cells. It plays a pivotal role in the regulation and activation of the antigen presenting cells. It has been found that clustering (or oligomerization) of CD40L is necessary for its effective activation of its cognate CD40 receptor on these immune cells. The use of anti-CD40 agonist antibodies has shown promise in cancer immunotherapy and vaccine adjuvants. However, dose limiting toxicities related to Fc receptor cross-linking of anti-CD40 agonist antibodies are a major issue currently being addressed in clinical trials.

Studies have shown that ligation of CD40 at the dendritic cell surface with CD40L-expressing CD4+ helper T cells is crucial for priming the MHC class I-antigen peptide complex to CD8+ cytotoxic T cells. CD8+ cytotoxic T cell priming in the absence of CD4+ helper T cells through binding to the MHC class II-antigen peptide complex on dendritic cells can cause impaired proliferative and cytotoxic capacities, a phenomenon described as “T cell exhaustion”. Ribas, et al. have reported that CD40 cross-linking can bypass the absolute requirement for CD4+ helper T cells during immunization with melanoma antigen gene-modified dendritic cells. See Ribas A, et al., “CD40 cross-linking bypasses the absolute requirement for CD4 T cells during immunization with melanoma antigen gene-modified dendritic cells”, Cancer Res. 2001 Dec. 15; 61(24):8787-93. PMID: 11751400.

Thus, there is a need for a non-Fc receptor-binding anti-CD40 agonist antibody or CD40 ligands that cluster CD40 receptors on the antigen presenting cells and that can reach the downstream cell signaling threshold of immune-stimulating events.

### SUMMARY

Disclosed embodiments solve these and other problems by providing multimeric CD40 ligands configured to cluster CD40 receptors on antigen presenting cells, thereby reaching a downstream cell signaling threshold of immune-stimulating events. Disclosed embodiments are directed to novel dendritic cell-based cancer vaccine therapies that resolve the issue of CD8+ T cell exhaustion caused by lacking CD4+ helper T during the dendritic cell activation or licensing process, using a soluble multimeric antigenic peptide-bearing CD40L fusion protein that can simultaneously activate dendritic cells and prime the MHC class I-antigen peptide complex to CD8+ T cells.

According to a first embodiment, there is provided a method for producing a multimeric fusion protein. The method comprises expressing in a mammalian cell a nucleic acid coding for an amino acid sequence comprising, in an N-terminal to C-terminal direction, a signal peptide, optionally an antigenic peptide, a CH3 domain of human IgG₁, a (G-P-P)₁₀ collagen-like domain, and a TNF ligand superfamily extracellular domain, the extracellular domain being devoid of a coiled-coil trimerization motif, and allowing the polypeptides expressed in the mammalian cell from the nucleic acid to at least one of trimerize and hexamerize into one or more multimeric fusion proteins.

The mammalian cell may be an antigen presenting cell.

The antigenic peptide may comprise one selected from the group consisting of epidermal growth factor receptor variant III peptide (PEP3), chicken ovalbumin (257-264) antigen peptide (OVA), and idiotypic antibody peptide derived from a BALB/c B cell lymphoma line A20 (A20ID).

The TNF ligand superfamily extracellular domain may comprise CD40L.

The antigenic peptide may form a peptide-major histocompatibility complex (MHC) protein complex on surfaces of an antigen presenting cell.

The antigenic peptide-MHC protein complex may be configured to engage T cell receptors on antigen peptide-specific T cells to stimulate an immune response.

The TNF ligand superfamily extracellular domain may comprise CD137L.

The antigen presenting cell may comprise a dendritic cell.

A nucleotide sequence of the nucleic acid may be SEQ ID NO: 4, SEQ ID NO: 6, or SEQ ID NO: 8, or a sequence having at least 90% sequence identity to the nucleotide sequence.

According to another embodiment, there is provided a method for producing a multimeric fusion protein, the method comprising expressing in an antigen presenting cell a nucleic acid coding for an amino acid sequence set forth in SEQ ID NO: 3, SEQ ID NO: 5, or SEQ ID NO: 7, or an amino acid sequence having at least 90% sequence identity to the amino acid sequence set forth in SEQ ID NO: 3, SEQ ID NO: 5, or SEQ ID NO: 7.

According to another embodiment, there is provided a soluble multimeric fusion protein comprising, from its N-terminus to C-terminus, optionally an antigenic peptide, a CH3 domain of human IgG₁, a (G-P-X1)₁₀ collagen-like domain, wherein X1 comprises P or O, and a TNF ligand superfamily extracellular domain, wherein the soluble multimeric fusion protein has a hexameric structure.

According to another embodiment, there is provided an isolated nucleic acid encoding the soluble multimeric fusion protein.

According to another embodiment, there is provided an isolated expression vector comprising the nucleic acid.

According to another embodiment, there is provided an isolated antigen presenting cell comprising the expression vector.

According to another embodiment, there is provided an isolated nucleic acid consisting of a nucleic acid coding for an amino acid sequence set forth in SEQ ID NO: 3, SEQ ID NO: 5, or SEQ ID NO: 7, or an amino acid sequence having at least 90% sequence identity to the amino acid sequence set forth in SEQ ID NO: 3, SEQ ID NO: 5, or SEQ ID NO: 7.

According to another embodiment, there is provided a pharmaceutical composition comprising a nucleic acid coding for the soluble multimeric fusion protein described above.

The pharmaceutical composition may further comprise a pharmaceutically acceptable carrier, excipient, or diluent.

The pharmaceutical composition may be used in treatment of cancer.

According to another embodiment, there is provided a method for treatment of cancer, the method comprising administering to a subject in need thereof the pharmaceutical composition described above.

The administering step may comprise delivering the pharmaceutical composition to dendritic cells of the subject ex vivo via transfection.

The administering step may comprise delivering the pharmaceutical composition to dendritic cells of the subject in vivo via a lipid nanoparticle.

The cancer may be at least one selected from the group consisting of lymphoma, breast cancer, lung cancer, colon cancer, rectal cancer, prostate cancer, melanoma, brain cancer, spinal cord cancer, ovarian cancer, pancreatic cancer, uterine cancer, and kidney cancer.

According to another embodiment, there is provided a method for controlling an immune response, the method comprising delivering the pharmaceutical composition described above to dendritic cells of a lymphatic system.

The pharmaceutical composition may activate CD40 on the dendritic cells and induce CD8+ T cells into memory cells through MHC class I antigenic peptide presentation.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIG. 1 is a schematic representation of direct T-cell priming and activation with mRNA-transfected dendritic cells according to an embodiment.

FIG. 2 depicts a schematic representation of different structural formats of CD40L fusion proteins. Format A: PepCSCD40L; Format B: PepCS6CD40L; Format C: CD40LCS6Pep; Format D: CS6-TNFL. Pep represents an antigenic peptide; ECD represents extracellular domain; CS is a collagen-like scaffold peptide, (GPP)₁₀; CS6 comprises the CH3 domain of human IgG₁, a short linker, and the collagen-like peptide, (GPP)₁₀. TNFL represents TNF ligand superfamily.

FIG. 3 depicts domain structure organization and purification of the recombinant CD40L fusion protein, PEP3CSCD40L. (A) Schematic representation of PEP3CSCD40L protein domains. The signal peptide (SP), epidermal growth factor receptor variant III (EGFRvIII) peptide (PEP3), collagen-like scaffold peptide (CS), the extracellular domain of CD40L, and the C-terminal affinity tag are illustrated. The amino acid sequence of PEP3 is shown. (B) SDS-PAGE analysis of PEP3CSCD40L protein purified from culture media by Strep-Tactin XT Superflow column. Lane 1: Bench Mark; Lane 2: PEP3CSCD40L (non-reduced); Lane 3: PEP3CSCD40L (reduced). The monomer and trimer of proteins are depicted and marked by arrows.

FIG. 4 depicts domain structure organization and purification of the recombinant CD40L fusion protein, PEP3CS6CD40L. (A) Schematic representation of PEP3CS6CD40L protein domains. The signal peptide (SP), epidermal growth factor receptor variant III (EGFRvIII) peptide (PEP3), CS6 domain consisting of the CH3 domain of human IgG₁ (CH3) and a (GPP)₁₀ collagen-like scaffold peptide, the extracellular domain of CD40L, and the C-terminal affinity tag are illustrated. The amino acid sequence of PEP3 is shown. (B) SDS-PAGE analysis of PEP3CS6CD40L and PEP3CSCD40L proteins purified from culture media by Strep-Tactin XT Superflow columns. Lane 1: Bench Mark; Lane 2: PEP3CS6CD40L (non-reduced); Lane 3: PEP3CS6CD40L (reduced); Lane 4: PEP3CSCD40L (non-reduced); Lane 5: PEP3CSCD40L (reduced). The monomer, trimer and hexamer of proteins are depicted and marked by arrows.

FIG. 5 depicts a size exclusion (SEC)-HPLC analysis of the purified PEP3CS6CD40L and PEP3CSCD40L proteins. Twenty micrograms each of the purified PEP3CS6CD40L and PEP3CSCD40L proteins were injected into a SEC-HPLC column (Zenix-C SEC-300) and absorbance at 280 nm were monitored. The representative hexameric and trimeric CD40L fusion proteins of PEP3CS6CD40L and PEP3CSCD40L are depicted, respectively. Elution peaks containing biotin in the sample buffer after Strep-Tactin XT column were indicated.

FIG. 6 depicts domain structure organization and purification of the hexameric CD40L fusion proteins. (A) Schematic representation of PepCS6CD40L protein domains, in which Pep denotes an antigenic peptide. The signal peptide (SP), the idiotypic antibody peptide derived from a BALB/c B cell lymphoma line A20 (A20ID) or a chicken ovalbumin (257-264) antigen peptide (OVA), CS6 domain consisting of the human CH3 domain of IgG₁ (CH3) and a (GPP)₁₀ collagen-like scaffold peptide, the extracellular domain of CD40L, and the C-terminal affinity tag are illustrated. The amino acid sequences of A20ID and OVA peptides are shown. (B) SDS-PAGE analysis of A20IDCS6CD40L and OVACS6CD40L proteins purified from culture media by Strep-Tactin XT Superflow columns. Lane 1: Bench Mark; Lane 2: A20IDCS6CD40L (non-reduced); Lane 3: A20IDCS6CD40L (reduced); Lane 4: OVACS6CD40L (non-reduced); Lane 5: OVACS6CD40L (reduced). The monomer, and hexamer of proteins are depicted and marked by arrows.

FIG. 7 depicts domain structure organization and purification of the hexameric TNF ligand family fusion proteins of mouse CD40L (CS6CD40L) and human CD137L (CS6hCD137L). (A) Schematic representation of CS6CD40L and CS6hCD137L, consisting of the human CH3 domain of IgG₁ (CH3) and a (GPP)₁₀ collagen-like scaffold peptide, the extracellular domain of mouse CD40L and the human CD137L, respectively. The C-terminal affinity tag is indicated for purification purpose. (B) SDS-PAGE analysis of CS6CD40L and CS6hCD137L proteins purified from culture media by Strep-Tactin XT Superflow columns. Lane 1: Bench Mark; Lane 2: CS6CD40L (non-reduced); Lane 3: CS6CD40L (reduced); Lane 4: CS6hCD137L (non-reduced); Lane 5: CS6hCD137L (reduced). The monomer, and hexamer of proteins are depicted and marked by arrows.

FIG. 8 depicts a CD40 activation assay by different CD40 agonist proteins, including PEP3CSCD40L (Δ), PEP3CS6CD40L (▴), and anti-CD40 monoclonal antibody (clone G28.5) (□). Assays were performed using a CD40 cell-based reporter system to evaluate CD40 cell activation, which is quantified by measuring firefly luciferase activity.

FIG. 9 depicts a CD137 activation assay by different CD137 agonist proteins, including CS6hCD137L (●) and the anti-CD137 monoclonal antibody (Urelumab analog) (◯). Assays were performed using a CD137 cell-based reporter system to evaluate CD137 cell activation, which is quantified by measuring firefly luciferase activity.

FIG. 10 depicts expression of surface markers. (A) Expression Levels of the surface markers: MHC-II, CD40, and CD86 in MutuDC1940 cells after treating with the 1 μg/ml of recombinant mouse CD40L trimer, PEP3CSCD40L trimer and PEP3CS6CD40L hexamer for 24 h. (B) Quantitation of the relative expression levels, as determined by the mean fluorescence intensity, of the above three CD40L fusions, as determined by flow cytometry.

FIG. 11 depicts exogenous antigen presentation by dendritic cells using OVA²⁵⁷⁻²⁶⁴ antigen peptide and OVACS6CD40L hexamer protein with an anti-OVA-H2K^{b} monoclonal antibody, clone 25D1.16, which specifically reacts with OVA257-264 peptide bound to H-2K^{b} of MHC class I.

FIG. 12 depicts structural and functional characterization of the secretory CD40L fusion proteins after delivering their mRNAs into mammalian cells. (A) HEK293T cells were individually transfected with 1 μg of PEP3CSCD40L (lanes 2-4) and PEP3CS6CD40L (lanes 5-7) mRNAs using MessengerMax transfection reagent. Culture supernatants were collected at indicated post transfection days and were analyzed by immunoblotting with an EGFRvIII-specific monoclonal antibody. Mock represents cells treated with transfection reagent only. (B) The culture supernatants from the mock, PEP3CSCD40L and PEP3CS6CD40 mRNA-transfected HEK293T cells after three days post transfection were analyzed by a cell-based CD40 activation assay.

FIG. 13 depicts mean fluorescence intensity of flow cytometry of various embodiments. MutuDC1940 cells were transfected with the indicated amount of mRNAs coding for PEP3CS6CD40L hexamer for 24 h. Quantitation of the relative expression levels of the dendritic cell-activating surface markers, MHC-II, CD40, and CD86 was determined by the mean fluorescence intensity of flow cytometry. Mock represents cells treated with transfection reagent only. Data from three independent experiments is presented as mean SD. Statistical significance was determined using an unpaired two-tailed t-test (*p<0.05; ****p<0.0001; ns: not significant).

FIG. 14 depicts antigen presentation of various embodiments. Mock represents cells transfected with only transfection reagents (without RNA).

FIG. 15 depicts T-cell priming and activation of various embodiments. (A) Schematic representation of direct T-cell priming and activation with OVACS6CD40L mRNA-transfected dendritic cells. mRNAs coding for OVACS6CD40L are formulated with lipid nanoparticles and are delivered into dendritic cells. (B) MutuDC1940 cells were transfected with CS6CD40L and OVACS6CD40L mRNAs for 24 hours, followed by co-culturing with CD8+ T cell hybridoma RF33.70 cells for another 48 h. The IL-2 in the culture supernatants was quantitated by an IL-2 reporter cell assay kit. Mock represents cells treated with transfection reagent only. (C) MutuDC1940 cells were treated with 2 μg/ml of OVA²⁵⁷⁻²⁶⁴ peptide or 10 μg/ml of purified OVACS6CD40L protein for 24 hours, followed by co-culturing with CD8⁺ T cell hybridoma RF33.70 cells for another 48 h. The IL-2 in the culture supernatants was quantitated by an IL-2 reporter cell assay kit.

FIGS. 16A-16C depict a tumor cell test according to embodiments. (A) Schematic representation of the animal study design. mRNAs coding for PEP3CS6CD40L were formulated with lipid nanoparticles (in vivo-jetRNA®+) and were immunized in mice intravenously. PEP3-KLH protein mixed with complete Freund's adjuvant was immunized in mice subcutaneously. (B) Representative slices of brain tumor size by H&E staining. (C) Total tumor volume was calculated by the sum of tumor area in each slice multiplied by the thickness and gap per slice. The column height represents the mean±SEM, and each dot represents an individual mouse. Statistical significance was determined using an unpaired two-tailed t test (*p<0.05, ***p<0.001). Tumor incidence rate and growth inhibition (TGI) rates for each study group are presented in the right panel.

FIGS. 17A-17C depict the growth of E.G7-OVA tumors in mice according to embodiments. (A) Schematic representation of the animal study design. C57BL/6 mice were immunized on days −14, −7, and −1 with mRNAs coding for OVACS6CD40L formulated with lipid nanoparticles (in vivo-jetRNA®+) by i.v. injection, OVA peptide mixed with poly(I:C) adjuvant by i.p. injection and were immunized on days −14, and −1, or PBS (control) by i.v. injection on days −14, −7, and −1. (B) The average tumor volume in each group. Tumor volume of mice immunizing with PBS (●), peptide/poly(I:C) (▴), and mRNA-LNP (▪) are shown. (C) In the left panel, the average tumor volume in each group. One hundred and eighty days after first tumor inoculation, the eight mRNA-LNP immunized, tumor-free mice (▪) in (B) were rechallenged with 3×10⁵ of E.G7-OVA cells. Six naïve control mice (●) were implanted with the same amount of tumor cells. In the right panel, individual tumor growth curves of each mouse after tumor challenge. Results are shown as the mean±SEM. Statistical significance was determined using an unpaired two-tailed t test (***p<0.001). CR indicates complete rejection.

### DETAILED DESCRIPTION

In the following detailed description, for purposes of explanation, numerous specific details are set forth in order to provide a thorough understanding of the disclosed embodiments. It will be apparent, however, that one or more embodiments may be practiced without these specific details.

Disclosed embodiments are directed to multimeric fusion proteins, methods for producing a multimeric fusion protein, nucleic acids, expression vectors, antigen presenting cells, pharmaceutical compositions, methods for treatment of cancer, and methods for controlling an immune response.

Disclosed embodiments are applicable to any suitable antigen presenting cells including, but not limited to, dendritic cells, macrophages, B cells. For purposes of this disclosure, embodiments will be described with respect to dendritic cells. However, it will be understood that this disclosure is not intended to be limited to dendritic cells and that embodiments are applicable to other antigen presenting cells.

Moreover, disclosed embodiments are applicable to any suitable target of the immune system. For purposes of this disclosure, embodiments will be described with respect to the lymphatic system, including lymphoid organs or nodes, and lymphatic cells, in particular. Lymphatic structures have been shown to be well suited for stimulating robust immune responses corresponding to vaccine or other therapeutic treatments.

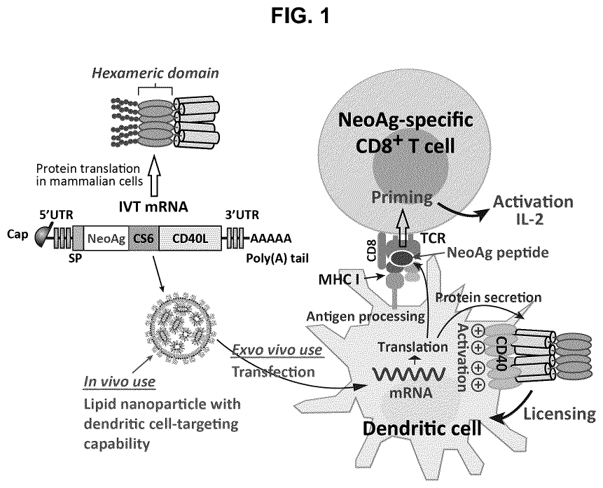

FIG. 1 illustrates development of a dendritic cell-based cancer vaccine using mRNA coding for a multimeric fusion protein, such as a soluble multimeric CD40L-bearing neoantigenic peptide fusion protein, according to embodiments.

### Multimeric Fusion Proteins

The multimeric fusion protein according to embodiments may include an antigenic peptide domain, such as a neoantigenic peptide fusion protein domain, a human IgG, a collagen-like scaffold peptide, and a TNF ligand protein. The multimeric fusion protein may have trimeric or hexameric structure. In preferred embodiments, the multimeric fusion protein has a hexameric structure. The hexameric structure may be formed by dimerization of the trimeric structure through a dimerization domain and a trimerization domain. In preferred embodiments, the dimerization domain is a human IgG, and the trimerization domain is a collagen-like scaffold peptide.

In embodiments, a stable trimer or hexamer structure may be formed, even in the presence of molecules which tend to form dimers. The formation of dimer of trimer requires carefully design of the two driving forces derived from the dimerization domain and the trimerization domain, as described in U.S. Pat. No. 8,669,350, which is hereby incorporated by reference in its entirety. For example, stabilizing the trimer structure of the multimeric fusion proteins may include, but is not limited to, increasing the repeat number of a G-P-P triplet and/or incorporating a trimerizing motif. This results in a thermally stable triple helical structure that drives the formation of a trimeric fusion protein, despite the presence of a strong dimerizing domain. Methods may involve destabilizing the dimerization power of the dimerization domains, while not interfering with the trimeric assembly of the fusion partners, in order to obtain pure trimeric Fc fusion proteins.

The antigenic peptide according to embodiments may be any suitable antigenic peptide. An antigenic peptide is a short segment of amino acids that can be recognized by the immune system, specifically by T-cell receptors. These peptides are derived from larger proteins or synthetic peptides. Antigenic peptides are typically presented on the surface of antigen-presenting cells (APCs), particularly dendritic cells, by major histocompatibility complex (MHC) molecules. This presentation is critical for the activation of T cells. In embodiments, the antigenic peptide may include, but is not limited to, an epidermal growth factor receptor variant III peptide (PEP3), an idiotypic antibody peptide derived from a BALB/c B cell lymphoma line A20 (A20ID) or a chicken ovalbumin (257-264) antigen peptide (OVA), for example.

In embodiments, the human IgG may be IgG₁, IgG₂, IgG₃, and/or IgG₄. In preferred embodiments, the human IgG comprises a CH3 domain of human IgG₁.

The CH3 domain of the human IgG₁ heavy chain constant region can form a homodimer as described by Ying et al. (2013). “Engineered Soluble Monomeric IgG1 CH3 Domain.” J Biol Chem. 288(35): 25154-25164, which is hereby incorporated by reference in its entirety.

The collagen-like scaffold peptide according to embodiments may be, for example, the multivalent Fab fragment with collagen-like peptide as described in U.S. Pat. No. 10,329,350, which is hereby incorporated by reference in its entirety. The collagen-like scaffold peptide according to embodiments may comprise at least one stretch of at least 5, at least 10, consecutive repeats of Gly-Pro-Pro or Gly-Pro-Hyp triplets. The collagen-like scaffold peptide may include a Gly-Pro-Pro or Gly-Pro-Hyp motif and/or other Gly-Xaa-Yaa motif, where Xaa and Yaa are any amino acid residues. The collagen-like scaffold peptide can also include a perfect repeating Gly-Xaa-Yaa triplet, interrupted by a short imperfection, in which the first position of Gly or the third position of Yaa residue is missing, found in many naturally occurring collagens and proteins containing collagen-like domains. This scaffold peptide allows for self-trimerization. A dimer of the trimeric fusion protein, i.e., a hexameric fusion protein, allows for superior clustering effect, as discussed herein. Without clustering adequate dendritic cell activation will not occur.

The assembled trimers of the embodiments include three monomers; a first, second and third multivalent antibody fragment. The assembled hexamers of the embodiments include dimers of the trimers; a first, second, third, fourth, fifth, and sixth multivalent antibody fragment. In one embodiment, the above-described first, second, third, fourth, fifth, and sixth multivalent antibody fragments are substantially identical, having at least 75% (e.g., any number between 75% and 100%, inclusive, e.g., 75%, 76% . . . 95%, 96%, 97%, 98%, or 99%) sequence identity to one another. A complex formed by three or six identical multivalent antibody fragments is a homotrimer or homohexamer, respectively. The three or six multivalent antibody fragments described herein can be functional equivalents. A “functional equivalent” refers to a polypeptide derivative of a common polypeptide, e.g., a protein having one or more point mutations, insertions, deletions, truncations, a fusion protein, or a combination thereof, and retaining substantially the ability to form a triple helix coil and the activity of the heterologous domain, such as binding to a ligand. In one embodiment there are three copies of a first monomer multivalent antibody fragment structure, and three copies of a second multivalent antibody fragment structure. In one embodiment there may be two copies of a first multivalent antibody fragment structure, two copies of a second multivalent antibody fragment structure, and two copies of a third polypeptide structure.

In the hexameric structure according to embodiments, each of the six monomer polypeptide sequences may be substantially identical. In one embodiment there are three copies of a first monomer fusion polypeptide sequence, and three copies of a second fusion polypeptide sequence. In one embodiment there may be two copies of a first fusion polypeptide sequence, two copies of a second fusion polypeptide sequence, and two copies of a third polypeptide sequence.

In embodiments, each monomer polypeptide may independently comprise (a) an extracellular domain of a TNF receptor family or a single domain antibody, (b) a collagen-like domain comprising at least 8 G-P-X1 blocks, wherein X1 may be P or O and a trimerizing motif, (c) optionally, a hinge region of IgG or a glycine linker, and (d) an Fc domain comprising the CH2 and CH3 regions of human IgG.

The percent identity can be determined, for example, by comparing sequence information using the GAP computer program, version 6.0 described by Devereux et al. (Nucl. Acids Res. 12:387, 1984) and available from the University of Wisconsin Genetics Computer Group (UWGCG). The GAP program utilizes the alignment method of Needleman and Wunsch (J. Mol. Biol. 48:443, 1970), as revised by Smith and Waterman (Adv. Appl. Math 2:482, 1981). The default parameters for the GAP program include: (1) a unary comparison matrix (containing a value of 1 for identities and 0 for non-identities) for nucleotides, and the weighted comparison matrix of Gribskov and Burgess, Nucl. Acids Res. 14:6745, 1986, as described by Schwartz and Dayhoff, eds., Atlas of Protein Sequence and Structure, National Biomedical Research Foundation, pp. 353-358, 1979; (2) a penalty of 3.0 for each gap and an additional 0.10 penalty for each symbol in each gap; and (3) no penalty for end gaps.

The TNF superfamily protein may include any one or more of the 19 structurally distinct ligands in the TNF superfamily, each of which are encoded by a unique gene, which can bind to 29 TNF receptor superfamily members. TNF proteins exhibit a characteristic protein fold, a shared TNF domain and trimeric structure, and are expressed as type II transmembrane proteins. Proteolytic cleavage of some membrane-anchored family members yields a soluble cytokine, as typified by the canonical family member TNF (formerly TNF-α) that was originally characterized for its role in tumor necrosis due to its ability to compromise endothelial integrity. TNF release also follows from the activation of neutrophils, CD4+ T cells, and innate lymphoid cells. The membrane-expressed TNF superfamily members have important actions on the ensuing adaptive immune response, and include CD40 ligand (CD40L), which activates antigen presenting cells, OX40L, which provide T-cell costimulation, FasL, CD27L, and CD30L, each of which regulates B- or T-lymphocyte homeostasis and apoptosis, and BLyS, which effects B-cell proliferation and differentiation and has been successfully targeted for SLE therapy.

For purposes of this disclosure, embodiments will be described with respect to CD40L. However, it will be understood that this disclosure is not intended to be limited to CD40L and that embodiments are applicable to other TNF ligand proteins.

CD40L is a T-helper cell membrane-bound trimer that activates CD40 on dendritic cells and induces cytotoxic CD8+ T cells into memory cells, as discussed herein. Moreover, without intending to be bound by theory, it is believed that CD40L may induce long-term B cell memory by clustering of multimeric antigen binders to B cell receptors. The clustering effect facilitated by the disclosed hexameric structure of the multimeric fusion protein is an important feature of the disclosed embodiments. The clustering of a multivalent soluble CD40L and CD40 on the surface of dendritic cells elicits a surprisingly strong downstream cellular signaling event, leading to dendritic cell maturation, induction of Th1 pathway by secreting IL-12, and promotion of CD8+ T cell activation through MHC class I antigenic peptide presentation.

Ligation of CD137 by its natural ligand, CD137L or 4-1BBL, activates CD8+ T cells and natural killer cells, resulting in enhanced cellular proliferation and survival, increased proinflammatory cytokine secretion, cytolytic function, and antibody-dependent cell-mediated cytotoxicity.

The ligand may bind the binding through intermolecular forces at a certain functional affinity. In one embodiment, the multimeric fusion protein has a functional affinity for its ligand of greater than 10⁻⁶ M. In one embodiment, multimeric fusion protein has a functional affinity for its ligand of greater than 10⁻⁸ M. In one embodiment, the multimeric fusion protein has a functional affinity for its ligand of greater than 10⁻¹⁰ M. In certain embodiments, the soluble trimeric or hexameric fusion protein has a functional affinity (or affinity) for its ligand between 10⁻⁷ M and 10⁻¹² M, between 10⁻⁸ M and 10⁻¹¹ M, between 10⁻⁷ M and 10⁻¹⁰ M, between 10⁻⁸ M and 10⁻¹⁰ M, and between 10⁻⁹ M and 10⁻¹⁰ M.

In embodiments, the CD40L may be soluble. A soluble protein is one that is soluble under physiological conditions. In one embodiment, the soluble trimeric or hexameric construct of the multimeric fusion protein is a secreted protein. A secreted fusion protein is one that is secreted by a cell. Secretion of a protein can be targeted by having a signal sequence or signal peptide on the polypeptide comprising the multimeric fusion protein domain.

The soluble multimeric CD40L can significantly reduce cytotoxicity caused by excessive expression of the transmembrane CD40L on the cell surface and compared to that of the transmembrane form or anti-CD40 antibodies. Multimeric CD40L fusions can differentiate the activated CD8+ T cells into memory CD8+ T cells. Multimeric CD40L-CD40 clustering elicits a strong downstream cellular signaling, leading to dendritic cell maturation bypasses the absolute requirement for CD4+ helper T cells.

The multimeric fusion protein may further include a signal peptide, a furin cleavage site, a TSMH affinity tag containing a thrombin cleavage site (LVPRGS), Strep-Tag II (WSHPQFEK), a c-myc epitope tag (EQKLISEEDL), and a polyhistidine tag (HHHHHH). In additional embodiments, a multimeric fusion protein may in include other conjugates such as, for example, a labeling agent (i.e., “a marking agent”), such as fluorescent agents or radioactive agents. Marker proteins include, but are not limited to, luciferase, green fluorescent protein, and enhanced green fluorescent protein. Multimeric fusion proteins of the present embodiments that include marker proteins can be used in diagnostic and molecular imaging. In embodiments, multimeric fusion proteins that include marker proteins or radioactive ions, or other fusion moieties, can be packaged in a kit including the multimeric fusion protein and other reagents necessary for imaging of specific molecules. These reagents can include, but are not limited to, reagents for the preparation of biological samples and reagents for the visualization of the marker protein.

In further embodiments, the multimeric fusion protein may be conjugated to a polymer. Such polymers include, but are not limited to, polyethylene glycol, polypropylene glycol, and polyoxyethylated polyol.

The multimeric fusion protein according to embodiments may further include conjugates or amino acid sequences or residues attached, appended, between, or among the above-described domains. The selective addition or removal of such conjugates or amino acid sequences or residues is encompassed by the disclosure. It will be understood that such selection may be employed to optimize functionality of the multimeric fusion protein under desired conditions.

Embodiments also encompass an isolated nucleic acid that contains a sequence encoding for the multimeric fusion protein or a complement of the sequence. A nucleic acid refers to a DNA molecule (e.g., a cDNA or genomic DNA), an RNA molecule (e.g., an mRNA), or a DNA or RNA analog. A DNA or RNA analog can be synthesized from nucleotide analogs. The nucleic acid molecule can be single-stranded or double-stranded, but in one embodiment is double-stranded DNA. An “isolated nucleic acid” is a nucleic acid, the structure of which is not identical to that of any naturally occurring nucleic acid or to that of any fragment of a naturally occurring genomic nucleic acid. The term therefore covers, for example, (a) a DNA which has the sequence of part of a naturally occurring genomic DNA molecule but is not flanked by both of the coding sequences that flank that part of the molecule in the genome of the organism in which it naturally occurs; (b) a nucleic acid incorporated into a vector or into the genomic DNA of a prokaryote or eukaryote in a manner such that the resulting molecule is not identical to any naturally occurring vector or genomic DNA; (c) a separate molecule such as a cDNA, a genomic fragment, a fragment produced by polymerase chain reaction (PCR), or a restriction fragment; and (d) a recombinant nucleotide sequence that is part of a hybrid gene, i.e., a gene encoding a fusion protein.

The nucleic acid described above can be used to express the polypeptide of embodiments. For this purpose, one can operatively link the nucleic acid to suitable regulatory sequences to generate an expression vector. A vector refers to a nucleic acid molecule capable of transporting another nucleic acid to which it has been linked. The vector can be capable of autonomous replication or integrate into a host DNA. Examples of a vector include a plasmid, cosmid, or viral vector. The vectors according to embodiments may include a nucleic acid in a form suitable for expression of the nucleic acid in a host cell. The vector includes one or more regulatory sequences operatively linked to the nucleic acid sequence to be expressed.

A “regulatory sequence” includes promoters, enhancers, and other expression control elements (e.g., polyadenylation signals). Regulatory sequences include those that direct constitutive expression of a nucleotide sequence, as well as tissue-specific regulatory and/or inducible sequences. The design of the expression vector can depend on such factors as the choice of the host cell to be transformed, the level of expression of protein desired, and the like. The expression vector can be introduced into dendritic cells to produce the polypeptide of embodiment. The dendritic cell may contain the above-described nucleic acid.

The sequences encoding the disclosed structures may also include nucleotide or protein sequences allowing for identification and purification. Such sequences can include restriction sites, tags, spacers, and other methods to purify or identify the nucleotide or protein sequence. Often such sequences are included in the nucleotide, and code for short amino acid sequences of 4-6 amino acids in length. They often appear in-between domains of the multivalent antibody fragment as artifacts, but do not materially affect the basic and novel characteristics of the invention.

To produce a multimeric fusion protein, one can culture a dendritic cell in a medium under conditions permitting expression of the polypeptide encoded by a nucleic acid, and purify the polypeptide from the cultured cell or the medium of the cell. Peptides containing collagen-like peptides can be difficult to purify if there are no affinity tags.

Loading of the mRNA nucleic acids into the dendritic cell may be achieved through any suitable means known in the art. In this regard, both in vivo and ex vivo applications of dendritic cell-based cancer vaccines are contemplated by this disclosure. Induction of Th1 response may occur through an endogenous mRNA expression pathway. As seen in FIG. 1, mRNA loading may occur through ex vivo transfection according to known methods in the art. The mRNA loading may also occur through in vivo applications via a lipid nanoparticle according to known methods in the art such as, for example, cationic lipid-mediated delivery, electroporation, and polymer-based nanoparticles.

In embodiments, the mRNA nucleic acids may be loaded through an endogenous protein expression pathway by delivery of a DNA or an mRNA coding for an MHC class I-specific peptide fused with a multivalent CD40L into dendritic cells, the secretory multivalent CD40L fusion protein can effectively activate the dendritic cells and, in the meantime, the dendritic cell-processed MHC class I-specific peptide can prime and activate its cognate CD8+ T cells. The CD40L-based dendritic cell adjuvant can be delivered into lymphoid organs using mRNA-nanoparticle approach, avoiding the systemic hepatotoxicity commonly associated with CD40-activating antibodies.

Disclosed embodiments also provide methods of treating, preventing or ameliorating the symptoms of cancer and T cell-mediated immunological diseases, such as autoimmune diseases, through the use of multimeric fusion proteins. Disclosed embodiments also provide methods of controlling an immune response.

In particular, the disclosed methods provide for administration of an mRNA coding for a multimeric fusion protein that are secreted outside of the dendritic cell, while at least some of the multimeric fusion proteins are degraded within the cell. The degradation of the fusion proteins within the cell are processed by the proteasomes. Following trimming by cytosolic proteases, the peptides are transported into the endoplasmic reticulum and loaded onto the cleft of MHC I complex and then transported peptide-loaded MHC I complex to the surface of the dendritic cell. The secreted intact multimeric fusion proteins are particularly well suited for achieving clustering of CD40 within the dendritic cells, following by activating the dendritic cells and priming the peptide-specific CD8+ T cells through ligation of the peptide-loaded MHC I complex, as discussed herein.

As used herein, cancer may include, but is not limited to, lymphoma, breast cancer, lung cancer, colon cancer, rectal cancer, prostate cancer, melanoma, brain cancer, spinal cord cancer, ovarian cancer, pancreatic cancer, uterine cancer, and/or kidney cancer.

As used herein, autoimmune disorders may refer to diseases in which the immune system mistakenly attacks and destroys healthy body tissue, thereby producing tissue injury. Autoimmune disorders include, but are not limited to, rheumatoid arthritis, juvenile rheumatoid arthritis, ankylosing spondylitis, multiple sclerosis, type 1 diabetes mellitus, inflammatory bowel diseases, systemic lupus erythematosus, mixed connective tissue disease, progressive systemic scleroderma, antiphospholipid syndrome, psoriasis, scleroderma, glomerulonephritis, dermatomyositis, Hashimoto's thyroiditis and Grave's disease.

Effector function may be demonstrated by apoptosis of tumor cells or phagocytosis and collapse of an antibody-coated particle through complement-dependent cytotoxicity (CDC), lysis of an antibody-coated target cell by crosslinking the antibody Fc fragment with the Fcγ receptors of an activated effector cell, such as natural killer cells, through antibody-dependent cell-mediated cytotoxicity (ADCC), cell membrane rupture, release of an inflammatory mediator, including IL-1α, IL-1β, IL-6, and TNFα, and control of immunoglobulin production.

T cell activation may be demonstrated by measuring T cell proliferation upon stimulation of T cells via antigen or agonistic antibodies to T cell receptor (TCR). TCR activation can lead to the initiation of signaling pathways including induction of specific protein tyrosine kinases (PTKs), breakdown of phosphatidylinositol 4,5-biphosphate (PIP2), activation of protein kinase C (PKC) and elevation of intracellular calcium ion concentration. These early events are transmitted to the nucleus and result in clonal expansion of T cells; upregulation of activation markers on the cell surface; differentiation into effector cells; induction of cytotoxicity or cytokine secretion such as IL-2; induction of apoptosis.

Cytokine release syndrome is manifested by, for example, headache, nausea, vomiting, fever, myalgias, arthralgias and shaking and may be caused by increased serum levels of, for example, IL-1α, IL-1β, IL-2, IL-6, IL-10, TNFα, and IFNγ.

The disclosed methods of treating such a disorder, e.g., by administering to a subject in need thereof an effective amount of the multimeric fusion protein to treat the disorder. Subjects to be treated can be identified as having, or being at risk for acquiring, a condition characterized by the disorder. This method can be performed alone or in conjunction with other drugs or therapy.

One embodiment is used for treating disorders caused by or exacerbated by T cell receptor/alloantigen interaction, and thus, regulate the T cell mediated toxicity associated with autoimmune disorders. In another embodiment, the treatments are used for modulating the biological activity of CD3, modulating the level of CD3 signaling, or modulating the T cell receptor/alloantigen interaction in a patient in need thereof. In an embodiment, the treatments decrease the level of unbound CD3 or CD3 signaling.

Activation of the cytotoxic T cell may occur via binding of the CD3 antigen as an effector antigen on the surface of the cytotoxic T cell by a multimeric fusion protein. Other lymphoid cell-associated effector antigens include the human CD16 antigen, NKG2D antigen, NKp46 antigen, CD2 antigen, CD28 antigen, CD25 antigen, CD64 antigen, and CD89 antigen. Binding to these effector antigens leads to activation of effector cells such as monocytes, neutrophilic granulocytes, and dendritic cells. These activated cells then exert a cytotoxic or an apoptotic effect on target cells.

The term “treating” is defined as administration of a composition to a subject with the purpose to cure, alleviate, relieve, remedy, prevent, or ameliorate a disorder, the symptom of the disorder, the disease state secondary to the disorder, a disorder which is exacerbated by the ligand of the multimeric fusion protein, or the predisposition toward the disorder. An “effective amount” is an amount of the composition that is capable of producing a medically desirable result, e.g., as described above, in a treated subject.

Disclosed embodiments also provide for a therapeutic composition (e.g., a composition containing the multimeric fusion protein) is administered to a subject. Generally, the complex is suspended in a pharmaceutically-acceptable carrier (e.g., physiological saline) and administered orally or by intravenous infusion, or injected or implanted subcutaneously, intramuscularly, intrathecally, intraperitoneally, intrarectally, intravaginally, intranasally, intragastrically, intratracheally, or intrapulmonarily. The pharmaceutical composition activates CD40 on the dendritic cells and induces CD8+ T cells into memory cells through MHC class I antigenic peptide presentation, as discussed herein.

The dosage required depends on the choice of the route of administration; the nature of the formulation; the nature of the subject's illness; the subject's size, weight, surface area, age, and sex; other drugs being administered; and the judgment of the attending physician.

Suitable dosages are in the range of 0.01-100.0 mg/kg. Suitable dosages are in the range of 0.01-100.0 mg/kg or more specifically 0.1-100, 0.1-75, 0.1-50, 0.1-25, 0.1-10, 0.5-100, 0.5-75, 0.5-50, 0.5-25, 0.5-10, 1-100, 1-75, 1-50, or 1-25 mg/kg. The dosages may include 1-10, 10-100, 10-75, 10-50, 10-25, 25-50, 50-75, 25-100, 25-50, 50-100, or 75-100 mg/kg. Or dosages can range from 1-2, 3-4, 5-6, 7-8, or 9-10 mg/kg.

Therapeutic compositions of the disclosed embodiments can be administered daily, one time, two times, or three times or more per week for between about 1 to 10 weeks, like between 2 to 8 weeks, or between about 3 to 7 weeks, and even for about 4, 5, or 6 weeks. Variations in the needed dosage are to be expected in view of the variety of compositions available and the different efficiencies of various routes of administration. For example, oral administration would be expected to require higher dosages than administration by intravenous injection. Variations in these dosage levels can be adjusted using standard empirical routines for optimization as is well understood in the art. Encapsulation of the composition in a suitable delivery vehicle (e.g., polymeric microparticles or implantable devices) may increase the efficiency of delivery, particularly for oral delivery.

The pharmaceutically acceptable carrier includes a solvent, a dispersion medium, a coating, an antibacterial and antifungal agent, and an isotonic and absorption delaying agent. Specifically, these agents can include saline solution, fixed oils, polyethylene glycols, glycerine, propylene glycol or other synthetic solvents; antibacterial agents such as benzyl alcohol or methyl parabens; antioxidants such as ascorbic acid or sodium bisulfite; chelating agents such as ethylenediaminetetraacetic acid; buffers such as acetates, citrates or phosphates; and agents for the adjustment of tonicity such as sodium chloride or dextrose. The pH of the pharmaceutical composition can be adjusted with acids or bases, such as hydrochloric acid or sodium hydroxide.

Disclosed embodiments also provide for a pharmaceutical composition that contains a pharmaceutically acceptable carrier and an effective amount of a multimeric fusion protein of the embodiments. The pharmaceutical composition can be used to treat the disorders listed above. The pharmaceutically acceptable carrier includes a solvent, a dispersion medium, a coating, an antibacterial and antifungal agent, and an isotonic and absorption delaying agent.

The pharmaceutical composition can be formulated into dosage forms for different administration routes utilizing conventional methods.

The efficacy of a composition of the disclosed embodiments can be evaluated both in vitro and in vivo. For in vivo studies, the composition can be injected into an animal (e.g., a mouse model) and its therapeutic effects are then accessed. Based on the results, an appropriate dosage range and administration route can be determined.

As used herein, the terms “directed against” and “specifically binds to” mean that the present fusion protein comprises an antibody domain, where the antibody or fragment of an antibody has a functional affinity of at least 10⁻⁶ M for its ligand.

### Structure of Multimeric Fusion Proteins

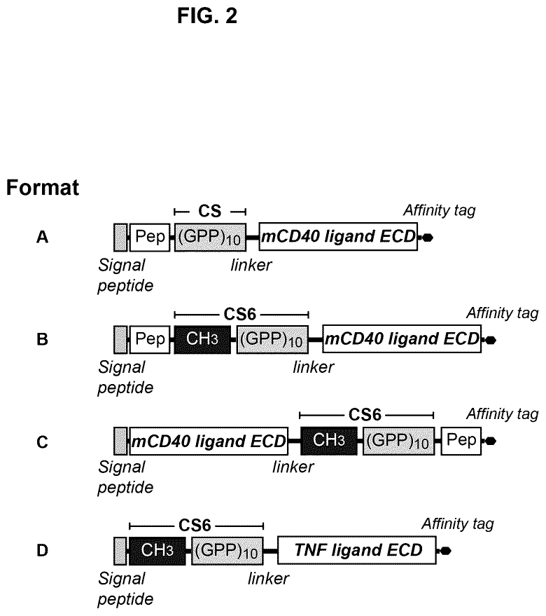

FIG. 2 is a schematic representation of different structural formats of multimeric fusion proteins, including the TNF family ligands. In both structural formats A and B of FIG. 2, the ECD of CD40 ligand is placed at the C-terminal and a peptide is placed at the N-terminal of the CS and CS6 domains, respectively. In structural format C, the ECD of CD40 ligand is placed at the N-terminal of the CS6 domain, followed by a peptide. In structural format D, the N-terminal peptide motif is removed and the ECD of either mouse CD40L or human CD137L is placed at the C-terminal of the CS6 domain, respectively. Placement of the CD40L on the C-terminal is important in disclosed embodiments in order to achieve proper secretion of the protein from the dendritic cell.

Sequences of multimeric fusion proteins according to embodiments may include the polypeptide sequence of PEP3CS6CD40L (SEQ ID NO: 3) and the cDNA sequence encoding it (SEQ ID NO: 4). The coding region of PEP3CS6CD40L, from N- to C-terminus, included a signal peptide (underline), a furin cleavage site (double-underline), the epidermal growth factor receptor variant III peptide (PEP3)—LEEKKGNYVVTDH (underline-boldface), the CH3 domain of human IgG₁ (bold italics), a (G-P-P)₁₀ collagen-like domain (boldface), an extracellular domain (ECD) of mouse CD40L (from amino acid residue Gly115 to Leu260, UniProt accession #P27548), which is devoid of the coiled-coil trimerization motif (italics), followed by a TSMH affinity tag containing a thrombin cleavage site (LVPRGS), Strep-Tag II (WSHPQFEK), a c-myc epitope tag (EQKLISEEDL), and a polyhistidine tag (HHHHHH).

SEQ ID NO: 3
METDTLLLWVLLLWVPGSTGDAAQPARRAKRSLEEKKGNYVVTDHASQ
PREPQVYTLPPSRDELTKNQVSLTCLVKGFYPSDIAVEWESNGQPENN
YKTTPPVLDSDGSFFLYSKLTVDKSRWQQGNVFSCSVMHEALHNHYTQ
KSLSLSPGAAAGSPGPPGPPGPPGPPGPPGPPGPPGPPGPPGPPGPSS
GGTGGDEDPQIAAHVVSEANSNAASVLQWAKKGYYTMKSNLVMLENGK
QLTVKREGLYYVYTQVTFCSNREPSSQRPFIVGLWLKPSSGSERILLK
AANTHSSSQLCEQQSVHLGGVFELQAGASVFVNVTEASQVIHRVGFSS
FGLLKLGSLVPRGSPGGSWSHPQFEKRRGGPEQKLISEEDLNSAVDGS
SHHHHHHSRGLE
SEQ ID NO: 4
ATGGAGACAGACACACTCCTGCTATGGGTACTGCTGCTCTGGGTTCCA
GGTTCCACTGGTGACGCGGCCCAGCCGGCCAGGCGCGCCAAACGATCA
CTGGAGGAGAAGAAGGGCAACTACGTGGTGACCGACCACGCTAGCCAG
CCCCGAGAACCACAGGTGTACACCCTGCCCCCATCCCGGGATGAGCTG
ACCAAGAACCAGGTCAGCCTGACCTGCCTGGTCAAAGGCTTCTATCCC
AGCGACATCGCCGTGGAGTGGGAGAGCAATGGGCAGCCGGAGAACAAC
TACAAGACCACGCCTCCCGTGCTGGACTCCGACGGCTCCTTCTTCCTC
TACAGCAAGCTCACCGTGGACAAGAGCAGGTGGCAGCAGGGGAACGTC
TTCTCATGCTCCGTGATGCATGAGGCTCTGCACAACCACTACACGCAG
AAGAGCCTCTCCCTGTCTCCGGGTGCGGCCGCTGGCAGCCCTGGGCCA
CCTGGTCCCCCAGGTCCTCCAGGACCCCCAGGGCCCCCAGGCCCCCCC
GGGCCGCCTGGACCCCCAGGGCCACCAGGCCCCCCAGGCCCTTCCTCT
GGCGGAACCGGTGGCGACGAAGATCCTCAAATCGCCGCACATGTCGTG
AGCGAGGCAAACAGTAACGCAGCAAGTGTTCTGCAATGGGCTAAAAAA
GGGTACTACACTATGAAATCCAACTTGGTAATGCTCGAAAACGGCAAA
CAGCTCACCGTCAAACGAGAAGGGCTCTATTATGTTTACACACAGGTA
ACTTTTTGTTCAAATAGGGAACCTTCTAGTCAACGTCCTTTCATTGTG
GGACTGTGGCTCAAGCCTAGTTCAGGCAGCGAGAGAATCTTGTTGAAA
GCAGCTAACACTCATTCCTCTTCACAATTGTGCGAGCAACAGTCAGTA
CACTTGGGAGGAGTATTTGAGTTGCAAGCTGGTGCCTCTGTTTTCGTT
AATGTGACCGAAGCCAGCCAGGTAATCCATCGAGTAGGTTTTTCTTCA
TTTGGGCTTCTCAAGCTCGGATCCCTGGTGCCGCGCGGCAGCCCTGGA
GGCTCCTGGAGCCACCCGCAGTTCGAAAAGCGTCGAGGAGGGCCAGAA
CAAAAACTCATCTCAGAAGAGGATCTGAATAGCGCCGTCGACGGCAGC
AGCCATCATCACCATCACCATAGCCGCGGACTCGAGTAA

Sequences of multimeric fusion proteins according to embodiments may include the polypeptide sequence of A20IDCS6CD40L (SEQ ID NO: 5) and the cDNA sequence encoding it (SEQ ID NO: 6). The coding region of A20IDCS6CD40L, from N- to C-terminus, included a signal peptide (underline), a furin cleavage site (double-underline), the idiotypic antibody peptide derived from a BALB/c B cell lymphoma line A20—DYWGQGTEL (underline-boldface), the CH3 domain of human IgG₁ (bold italics), a (G-P-P)₁₀ collagen-like domain (boldface), an extracellular domain (ECD) of mouse CD40L (from amino acid residue Gly115 to Leu260, UniProt accession #P27548), which is devoid of the coiled-coil trimerization motif (italics), followed by a TSMH affinity tag containing a thrombin cleavage site (LVPRGS), Strep-Tag II (WSHPQFEK), a c-myc epitope tag (EQKLISEEDL), and a polyhistidine tag (HHHHHH).

SEQ ID NO: 5
METDTLLLWVLLLWVPGSTGDAAQPARRAKRSDYWGQGTELASQPREP
QVYTLPPSRDELTKNQVSLTCLVKGFYPSDIAVEWESNGQPENNYKTT
PPVLDSDGSFFLYSKLTVDKSRWQQGNVFSCSVMHEALHNHYTQKSLS
LSPGAAAGSPGPPGPPGPPGPPGPPGPPGPPGPPGPPGPPGPSSGGTG
GDEDPQIAAHVVSEANSNAASVLQWAKKGYYTMKSNLVMLENGKQLTV
KREGLYYVYTQVTFCSNREPSSQRPFIVGLWLKPSSGSERILLKAANT
HSSSQLCEQQSVHLGGVFELQAGASVFVNVTEASQVIHRVGFSSFGLL
KLGSLVPRGSPGGSWSHPQFEKRRGGPEQKLISEEDLNSAVDGSSHHH
HHHSRGLE
SEQ ID NO: 6
ATGGAGACAGACACACTCCTGCTATGGGTACTGCTGCTCTGGGTTCCA
GGTTCCACTGGTGACGCGGCCCAGCCGGCCAGGCGCGCCAAACGATCA
GACTACTGGGGCCAAGGCACAGAACTAGCTAGCCAGCCCCGAGAACCA
CAGGTGTACACCCTGCCCCCATCCCGGGATGAGCTGACCAAGAACCAG
GTCAGCCTGACCTGCCTGGTCAAAGGCTTCTATCCCAGCGACATCGCC
GTGGAGTGGGAGAGCAATGGGCAGCCGGAGAACAACTACAAGACCACG
CCTCCCGTGCTGGACTCCGACGGCTCCTTCTTCCTCTACAGCAAGCTC
ACCGTGGACAAGAGCAGGTGGCAGCAGGGGAACGTCTTCTCATGCTCC
GTGATGCATGAGGCTCTGCACAACCACTACACGCAGAAGAGCCTCTCC
CTGTCTCCGGGTGCGGCCGCTGGCAGCCCTGGGCCACCTGGTCCCCCA
GGTCCTCCAGGACCCCCAGGGCCCCCAGGCCCCCCCGGGCCGCCTGGA
CCCCCAGGGCCACCAGGCCCCCCAGGCCCTTCCTCTGGCGGAACCGGT
GGCGACGAAGATCCTCAAATCGCCGCACATGTCGTGAGCGAGGCAAAC
AGTAACGCAGCAAGTGTTCTGCAATGGGCTAAAAAAGGGTACTACACT
ATGAAATCCAACTTGGTAATGCTCGAAAACGGCAAACAGCTCACCGTC
AAACGAGAAGGGCTCTATTATGTTTACACACAGGTAACTTTTTGTTCA
AATAGGGAACCTTCTAGTCAACGTCCTTTCATTGTGGGACTGTGGCTC
AAGCCTAGTTCAGGCAGCGAGAGAATCTTGTTGAAAGCAGCTAACACT
CATTCCTCTTCACAATTGTGCGAGCAACAGTCAGTACACTTGGGAGGA
GTATTTGAGTTGCAAGCTGGTGCCTCTGTTTTCGTTAATGTGACCGAA
GCCAGCCAGGTAATCCATCGAGTAGGTTTTTCTTCATTTGGGCTTCTC
AAGCTCGGATCCCTGGTGCCGCGCGGCAGCCCTGGAGGCTCCTGGAGC
CACCCGCAGTTCGAAAAGCGTCGAGGAGGGCCAGAACAAAAACTCATC
TCAGAAGAGGATCTGAATAGCGCCGTCGACGGCAGCAGCCATCATCAC
CATCACCATAGCCGCGGACTCGAGTAA

Sequences of multimeric fusion proteins according to embodiments may include the polypeptide sequence of OVACS6CD40L (SEQ ID NO: 7) and the cDNA sequence encoding it (SEQ ID NO: 8). The coding region of OVACS6CD40L, from N- to C-terminus, included a signal peptide (underline), a furin cleavage site (double-underline), a chicken ovalbumin antigen peptide—SIINFEKL (underline-boldface), the CH3 domain of human IgG₁ (bold italics), a (G-P-P)₁₀ collagen-like domain (boldface), an extracellular domain (ECD) of mouse CD40L (from amino acid residue Gly115 to Leu260, UniProt accession #P27548), which is devoid of the coiled-coil trimerization motif (italics), followed by a TSMH affinity tag containing a thrombin cleavage site (LVPRGS), Strep-Tag II (WSHPQFEK), a c-myc epitope tag (EQKLISEEDL), and a polyhistidine tag (HHHHHH).

SEQ ID NO: 7
METDTLLLWVLLLWVPGSTGDAAQPARRAKRSSIINFEKLASQPREPQ
VYTLPPSRDELTKNQVSLTCLVKGFYPSDIAVEWESNGQPENNYKTTP
PVLDSDGSFFLYSKLTVDKSRWQQGNVFSCSVMHEALHNHYTQKSLSL
SPGAAAGSPGPPGPPGPPGPPGPPGPPGPPGPPGPPGPPGPSSGGTGG
DEDPQIAAHVVSEANSNAASVLQWAKKGYYTMKSNLVMLENGKQLTVK
REGLYYVYTQVTFCSNREPSSQRPFIVGLWLKPSSGSERILLKAANTH
SSSQLCEQQSVHLGGVFELQAGASVFVNVTEASQVIHRVGFSSFGLLK
LGSLVPRGSPGGSWSHPQFEKRRGGPEQKLISEEDLNSAVDGSSHHHH
HHSRGLE
SEQ ID NO: 8
ATGGAGACAGACACACTCCTGCTATGGGTACTGCTGCTCTGGGTTCCA
GGTTCCACTGGTGACGCGGCCCAGCCGGCCAGGCGCGCCAAACGATCA
AGTATCATCAACTTTGAAAAGCTGGCTAGCCAGCCCCGAGAACCACAG
GTGTACACCCTGCCCCCATCCCGGGATGAGCTGACCAAGAACCAGGTC
AGCCTGACCTGCCTGGTCAAAGGCTTCTATCCCAGCGACATCGCCGTG
GAGTGGGAGAGCAATGGGCAGCCGGAGAACAACTACAAGACCACGCCT
CCCGTGCTGGACTCCGACGGCTCCTTCTTCCTCTACAGCAAGCTCACC
GTGGACAAGAGCAGGTGGCAGCAGGGGAACGTCTTCTCATGCTCCGTG
ATGCATGAGGCTCTGCACAACCACTACACGCAGAAGAGCCTCTCCCTG
TCTCCGGGTGCGGCCGCTGGCAGCCCTGGGCCACCTGGTCCCCCAGGT
CCTCCAGGACCCCCAGGGCCCCCAGGCCCCCCCGGGCCGCCTGGACCC
CCAGGGCCACCAGGCCCCCCAGGCCCTTCCTCTGGCGGAACCGGTGGC
GACGAAGATCCTCAAATCGCCGCACATGTCGTGAGCGAGGCAAACAGT
AACGCAGCAAGTGTTCTGCAATGGGCTAAAAAAGGGTACTACACTATG
AAATCCAACTTGGTAATGCTCGAAAACGGCAAACAGCTCACCGTCAAA
CGAGAAGGGCTCTATTATGTTTACACACAGGTAACTTTTTGTTCAAAT
AGGGAACCTTCTAGTCAACGTCCTTTCATTGTGGGACTGTGGCTCAAG
CCTAGTTCAGGCAGCGAGAGAATCTTGTTGAAAGCAGCTAACACTCAT
TCCTCTTCACAATTGTGCGAGCAACAGTCAGTACACTTGGGAGGAGTA
TTTGAGTTGCAAGCTGGTGCCTCTGTTTTCGTTAATGTGACCGAAGCC
AGCCAGGTAATCCATCGAGTAGGTTTTTCTTCATTTGGGCTTCTCAAG
CTCGGATCCCTGGTGCCGCGCGGCAGCCCTGGAGGCTCCTGGAGCCAC
CCGCAGTTCGAAAAGCGTCGAGGAGGGCCAGAACAAAAACTCATCTCA
GAAGAGGATCTGAATAGCGCCGTCGACGGCAGCAGCCATCATCACCAT
CACCATAGCCGCGGACTCGAGTAA

The amino acid and nucleotide sequences according to embodiments may further include amino acid sequences/residues and nucleotide sequences/residues attached, appended, between, or among the above-described domains so long as formats A and B of FIG. 2, i.e., the ECD of CD40 ligand is placed at the C-terminal and a peptide is placed at the N-terminal of the CS and CS6 domains, respectively, are preserved. The selective addition or removal of such sequences or residues is encompassed by the disclosure. It will be understood that such selection may be employed to optimize functionality of the multimeric fusion protein under desired conditions.

In some aspects, an isolated nucleic acid molecule comprising a nucleotide sequence that encodes a polypeptide having at least 85%, 86%, 87%, 88%, 89%, 90%, 91%, 92%, 93%, 94%, 95%, 96%, 97%, 98%, or 99% sequence identity to an amino acid sequence of any one of SEQ ID NOs: 3, 5, and 7.

In some aspects, the nucleotide sequence is a sequence having at least 85%, 86%, 87%, 88%, 89%, 90%, 91%, 92%, 93%, 94%, 95%, 96%, 97%, 98%, or 99% sequence identity to a nucleotide sequence of any one of SEQ ID NOs: 4, 6, and 8.

Signal peptide sequences may include:
(SEQ ID NO: 15)
METDTLLLWVLLLWVPGSTG
The signal peptide may be cleaved off during the
expression, assembly, and/or secretion process.
Furin cleavage site sequences may include:
(SEQ ID NO: 16)
RAKR
PEP3 sequences may include:
(SEQ ID NO: 17)
LEEKKGNYVVTDH
A20ID sequences may include:
(SEQ ID NO: 18)
DYWGQGTEL
OVA sequences may include:
(SEQ ID NO: 19)
SIINFEKL
The CH3 domain of human IgG₁ sequences may
include:
(SEQ ID NO: 20)
QPREPQVYTLPPSRDELTKNQVSLTCLVKGFYPSDIAVEWESNGQPE
NNYKTTPPVLDSDGSFFLYSKLTVDKSRWQQGNVFSCSVMHEALHNH
YTQKSLSLSPG
Collagen-like polypeptide sequences may include:
(SEQ ID NO: 21)
GPPGPPGPPGPPGPPGPPGPPGPPGPPGPP
CD40L sequences may include:
(SEQ ID NO: 22)
GDEDPQIAAHVVSEANSNAASVLQWAKKGYYTMKSNLVMLENGKQLT
VKREGLYYVYTQVTFCSNREPSSQRPFIVGLWLKPSSGSERILLKAA
NTHSSSQLCEQQSVHLGGVFELQAGASVFVNVTEASQVIHRVGFSSF
GLLKL

The specific examples below are to be construed as merely illustrative, and not limiting of the remainder of the disclosure in any way whatsoever.

### EXAMPLES

### Example 1

### Construction of Plasmids Expressing the Soluble Multimeric C D40 Ligand Fusion Proteins—PEP3CSCD40L, PEP3CS6CD40L, A20IDCS6CD40L, OVACS6CD40L, and CD40LCS6OVA

Listed below are the polypeptide sequence of PEP3CSCD40L (SEQ ID NO: 1) and the cDNA sequence encoding it (SEQ ID NO: 2). The coding region of PEP3CSCD40L, from N- to C-terminus, included a signal peptide (underline), a furin cleavage site (double-underline), the epidermal growth factor receptor variant III peptide (PEP3)—LEEKKGNYVVTDH (underline-boldface), a (G-P-P)₁₀ collagen-like domain (boldface), an extracellular domain (ECD) of mouse CD40L (from amino acid residue Gly115 to Leu260, UniProt accession #P27548), which is devoid of the coiled-coil trimerization motif (italics), followed by a TSMH affinity tag containing a thrombin cleavage site (LVPRGS), Strep-Tag II (WSHPQFEK), a c-myc epitope tag (EQKLISEEDL), and a polyhistidine tag (HHHHHH). The cDNA synthetic sequence (SEQ ID NO: 2) of the above polypeptide chain was codon-optimized by overlapping PCR and the PCR product was subcloned into a modified expression vector of pSecTag2/Hygro (Cat. No. V90020, Thermo Fisher Scientific Inc.), in which the original C-terminal affinity tag sequence was replaced with the TSMH affinity tag for detection and purification purposes.

SEQ ID NO: 1
METDTLLLWVLLLWVPGSTGDAAQPARRAKRSLEEKKGNYVVTDHAAA
GSPGPPGPPGPPGPPGPPGPPGPPGPPGPPGPPGPSSGGTGGDEDPQI
AAHVVSEANSNAASVLQWAKKGYYTMKSNLVMLENGKQLTVKREGLYY
VYTQVTFCSNREPSSQRPFIVGLWLKPSSGSERILLKAANTHSSSQLC
EQQSVHLGGVFELQAGASVFVNVTEASQVIHRVGFSSFGLLKLGSLVP
RGSPGGSWSHPQFEKRRGGPEQKLISEEDLNSAVDGSSHHHHHHSRGL
E
SEQ ID NO: 2
ATGGAGACAGACACACTCCTGCTATGGGTACTGCTGCTCTGGGTTCCA
GGTTCCACTGGTGACGCGGCCCAGCCGGCCAGGCGCGCCAAACGATCA
CTGGAGGAGAAGAAGGGCAACTACGTGGTGACCGACCACGCGGCCGCT
GGCAGCCCTGGGCCACCTGGTCCCCCAGGTCCTCCAGGACCCCCAGGG
CCCCCAGGCCCCCCCGGGCCGCCTGGACCCCCAGGGCCACCAGGCCCC
CCAGGCCCTTCCTCTGGCGGAACCGGTGGCGACGAAGATCCTCAAATC
GCCGCACATGTCGTGAGCGAGGCAAACAGTAACGCAGCAAGTGTTCTG
CAATGGGCTAAAAAAGGGTACTACACTATGAAATCCAACTTGGTAATG
CTCGAAAACGGCAAACAGCTCACCGTCAAACGAGAAGGGCTCTATTAT
GTTTACACACAGGTAACTTTTTGTTCAAATAGGGAACCTTCTAGTCAA
CGTCCTTTCATTGTGGGACTGTGGCTCAAGCCTAGTTCAGGCAGCGAG
AGAATCTTGTTGAAAGCAGCTAACACTCATTCCTCTTCACAATTGTGC
GAGCAACAGTCAGTACACTTGGGAGGAGTATTTGAGTTGCAAGCTGGT
GCCTCTGTTTTCGTTAATGTGACCGAAGCCAGCCAGGTAATCCATCGA
GTAGGTTTTTCTTCATTTGGGCTTCTCAAGCTCGGATCCCTGGTGCCG
CGCGGCAGCCCTGGAGGCTCCTGGAGCCACCCGCAGTTCGAAAAGCGT
CGAGGAGGGCCAGAACAAAAACTCATCTCAGAAGAGGATCTGAATAGC
GCCGTCGACGGCAGCAGCCATCATCACCATCACCATAGCCGCGGACTC
GAGTAA

PEP3CS6CD40L was constructed according to SEQ ID NO: 3. The cDNA synthetic sequence (SEQ ID NO: 4) of the above polypeptide chain was codon-optimized by overlapping PCR and the PCR product was subcloned into a modified expression vector of pSecTag2/Hygro (Cat. No. V90020, Thermo Fisher Scientific Inc.), in which the original C-terminal affinity tag sequence was replaced with the TSMH affinity tag for detection and purification purposes.

A20IDCS6CD40L was constructed according to SEQ ID NO: 5. The cDNA synthetic sequence (SEQ ID NO: 6) of the above polypeptide chain was codon-optimized by overlapping PCR and the PCR product was subcloned into a modified expression vector of pSecTag2/Hygro (Cat. No. V90020, Thermo Fisher Scientific Inc.), in which the original C-terminal affinity tag sequence was replaced with the TSMH affinity tag for detection and purification purposes.

OVACS6CD40L was constructed according to SEQ ID NO: 7. The cDNA synthetic sequence (SEQ ID NO: 8) of the above polypeptide chain was codon-optimized by overlapping PCR and the PCR product was subcloned into a modified expression vector of pSecTag2/Hygro (Cat. No. V90020, Thermo Fisher Scientific Inc.), in which the original C-terminal affinity tag sequence was replaced with the TSMH affinity tag for detection and purification purposes.

Listed below are the polypeptide sequence of CD40LCS6OVA (SEQ ID NO: 9) and the cDNA sequence encoding it (SEQ ID NO: 10). The coding region of CD40LCS6OVA, from N- to C-terminus, included a signal peptide (underline), an extracellular domain (ECD) of mouse CD40L (from amino acid residue Gly115 to Leu260, UniProt accession #P27548), which is devoid of the coiled-coil trimerization motif (italics), the CH3 domain of human IgG₁ (bold italics), a (G-P-P)₁₀ collagen-like domain (boldface), a chicken ovalbumin antigen peptide—SIINFEKL (underline-boldface), followed by a TSMH affinity tag containing a thrombin cleavage site (LVPRGS), Strep-Tag II (WSHPQFEK), a c-myc epitope tag (EQKLISEEDL), and a polyhistidine tag (HHHHHH). The cDNA synthetic sequence (SEQ ID NO: 10) of the above polypeptide chain was codon-optimized by overlapping PCR and the PCR product was subcloned into a modified expression vector of pSecTag2/Hygro (Cat. No. V90020, Thermo Fisher Scientific Inc.), in which the original C-terminal affinity tag sequence was replaced with the TSMH affinity tag for detection and purification purposes.

SEQ ID NO: 9
METDTLLLWVLLLWVPGSTGDAAQPARRAGDEDPQIAAHVVSEANSNA
ASVLQWAKKGYYTMKSNLVMLENGKQLTVKREGLYYVYTQVTFCSNRE
PSSQRPFIVGLWLKPSSGSERILLKAANTHSSSQLCEQQSVHLGGVFE
LQAGASVFVNVTEASQVIHRVGFSSFGLLKLRTGPSSGGASQPREPQV
YTLPPSRDELTKNQVSLTCLVKGFYPSDIAVEWESNGQPENNYKTTPP
VLDSDGSFFLYSKLTVDKSRWQQGNVFSCSVMHEALHNHYTQKSLSLS
PGAAAGSPGPPGPPGPPGPPGPPGPPGPPGPPGPPGPPGPSSGGTGSI
INFEKLGSLVPRGSPGGSWSHPQFEKRRGGPEQKLISEEDLNSAVDGS
SHHHHHHSRGLE
SEQ ID NO: 10
ATGGAGACAGACACACTCCTGCTATGGGTACTGCTGCTCTGGGTTCCA
GGTTCCACTGGTGACGCGGCCCAGCCGGCCAGGCGCGCCGGCGACGAA
GATCCTCAAATCGCCGCACATGTCGTGAGCGAGGCAAACAGTAACGCA
GCAAGTGTTCTGCAATGGGCTAAAAAAGGGTACTACACTATGAAATCC
AACTTGGTAATGCTCGAAAACGGCAAACAGCTCACCGTCAAACGAGAA
GGGCTCTATTATGTTTACACACAGGTAACTTTTTGTTCAAATAGGGAA
CCTTCTAGTCAACGTCCTTTCATTGTGGGACTGTGGCTCAAGCCTAGT
TCAGGCAGCGAGAGAATCTTGTTGAAAGCAGCTAACACTCATTCCTCT
TCACAATTGTGCGAGCAACAGTCAGTACACTTGGGAGGAGTATTTGAG
TTGCAAGCTGGTGCCTCTGTTTTCGTTAATGTGACCGAAGCCAGCCAG
GTAATCCATCGAGTAGGTTTTTCTTCATTTGGGCTTCTCAAGCTCCGT
ACGGGCCCTTCCTCTGGCGGAGCTAGCCAGCCCCGAGAACCACAGGTG
TACACCCTGCCCCCATCCCGGGATGAGCTGACCAAGAACCAGGTCAGC
CTGACCTGCCTGGTCAAAGGCTTCTATCCCAGCGACATCGCCGTGGAG
TGGGAGAGCAATGGGCAGCCGGAGAACAACTACAAGACCACGCCTCCC
GTGCTGGACTCCGACGGCTCCTTCTTCCTCTACAGCAAGCTCACCGTG
GACAAGAGCAGGTGGCAGCAGGGGAACGTCTTCTCATGCTCCGTGATG
CATGAGGCTCTGCACAACCACTACACGCAGAAGAGCCTCTCCCTGTCT
CCGGGTGCGGCCGCTGGCAGCCCTGGGCCACCTGGTCCCCCAGGTCCT
CCAGGACCCCCAGGGCCCCCAGGCCCCCCCGGGCCGCCTGGACCCCCA
GGGCCACCAGGCCCCCCAGGCCCTTCCTCTGGCGGAACCGGTAGTATC
ATCAACTTTGAAAAGCTGGGATCCCTGGTGCCGCGCGGCAGCCCTGGA
GGCTCCTGGAGCCACCCGCAGTTCGAAAAGCGTCGAGGAGGGCCAGAA
CAAAAACTCATCTCAGAAGAGGATCTGAATAGCGCCGTCGACGGCAGC
AGCCATCATCACCATCACCATAGCCGCGGACTCGAGTAA

Listed below are the polypeptide sequence of CS6CD40L (SEQ ID NO: 11) and the cDNA sequence encoding it (SEQ ID NO: 12). The coding region of CS6CD40L, from N- to C-terminus, included a signal peptide (underline), a furin cleavage site (double-underline), the CH3 domain of human IgG₁ (bold italics), a (G-P-P)₁₀ collagen-like domain (boldface), an extracellular domain (ECD) of mouse CD40L (from amino acid residue Gly115 to Leu260, UniProt accession #P27548), which is devoid of the coiled-coil trimerization motif (italics), followed by a TSMH affinity tag containing a thrombin cleavage site (LVPRGS), Strep-Tag II (WSHPQFEK), a c-myc epitope tag (EQKLISEEDL), and a polyhistidine tag (HHHHHH). The cDNA synthetic sequence (SEQ ID NO: 12) of the above polypeptide chain was codon-optimized by overlapping PCR and the PCR product was subcloned into a modified expression vector of pSecTag2/Hygro (Cat. No. V90020, Thermo Fisher Scientific Inc.), in which the original C-terminal affinity tag sequence was replaced with the TSMH affinity tag for detection and purification purposes.

SEQ ID NO: 11
METDTLLLWVLLLWVPGSTGDAAQPARRAKRSQPREPQVYTLPPSRDE
LTKNQVSLTCLVKGFYPSDIAVEWESNGQPENNYKTTPPVLDSDGSFF
LYSKLTVDKSRWQQGNVFSCSVMHEALHNHYTQKSLSLSPGAAAGSPG
PPGPPGPPGPPGPPGPPGPPGPPGPPGPPGPSSGGTGGDEDPQIAAHV
VSEANSNAASVLQWAKKGYYTMKSNLVMLENGKQLTVKREGLYYVYTQ
VTFCSNREPSSQRPFIVGLWLKPSSGSERILLKAANTHSSSQLCEQQS
VHLGGVFELQAGASVFVNVTEASQVIHRVGFSSFGLLKLGSLVPRGSP
GGSWSHPQFEKRRGGPEQKLISEEDLNSAVDGSSHHHHHHSRGLE
SEQ ID NO: 12
ATGGAGACAGACACACTCCTGCTATGGGTACTGCTGCTCTGGGTTCCA
GGTTCCACTGGTGACGCGGCCCAGCCGGCCAGGCGCGCCAAACGATCA
CAGCCCCGAGAACCACAGGTGTACACCCTGCCCCCATCCCGGGATGAG
CTGACCAAGAACCAGGTCAGCCTGACCTGCCTGGTCAAAGGCTTCTAT
CCCAGCGACATCGCCGTGGAGTGGGAGAGCAATGGGCAGCCGGAGAAC
AACTACAAGACCACGCCTCCCGTGCTGGACTCCGACGGCTCCTTCTTC
CTCTACAGCAAGCTCACCGTGGACAAGAGCAGGTGGCAGCAGGGGAAC
GTCTTCTCATGCTCCGTGATGCATGAGGCTCTGCACAACCACTACACG
CAGAAGAGCCTCTCCCTGTCTCCGGGTGCGGCCGCTGGCAGCCCTGGG
CCACCTGGTCCCCCAGGTCCTCCAGGACCCCCAGGGCCCCCAGGCCCC
CCCGGGCCGCCTGGACCCCCAGGGCCACCAGGCCCCCCAGGCCCTTCC
TCTGGCGGAACCGGTGGCGACGAAGATCCTCAAATCGCCGCACATGTC
GTGAGCGAGGCAAACAGTAACGCAGCAAGTGTTCTGCAATGGGCTAAA
AAAGGGTACTACACTATGAAATCCAACTTGGTAATGCTCGAAAACGGC
AAACAGCTCACCGTCAAACGAGAAGGGCTCTATTATGTTTACACACAG
GTAACTTTTTGTTCAAATAGGGAACCTTCTAGTCAACGTCCTTTCATT
GTGGGACTGTGGCTCAAGCCTAGTTCAGGCAGCGAGAGAATCTTGTTG
AAAGCAGCTAACACTCATTCCTCTTCACAATTGTGCGAGCAACAGTCA
GTACACTTGGGAGGAGTATTTGAGTTGCAAGCTGGTGCCTCTGTTTTC
GTTAATGTGACCGAAGCCAGCCAGGTAATCCATCGAGTAGGTTTTTCT
TCATTTGGGCTTCTCAAGCTCGGATCCCTGGTGCCGCGCGGCAGCCCT
GGAGGCTCCTGGAGCCACCCGCAGTTCGAAAAGCGTCGAGGAGGGCCA
GAACAAAAACTCATCTCAGAAGAGGATCTGAATAGCGCCGTCGACGGC
AGCAGCCATCATCACCATCACCATAGCCGCGGACTCGAGTAA

### Example 2

### Construction of a Plasmid Expressing the Soluble Multimeric CD137 Ligand Fusion Proteins—CS6hCD137L

Listed below are the polypeptide sequence of CS6hCD137L (SEQ ID NO: 13) and the cDNA sequence encoding it (SEQ ID NO: 14). The coding region of CS6hCD137L, from N- to C-terminus, included a signal peptide (underline), a furin cleavage site (double-underline), the CH3 domain of human IgG₁ (bold italics), a (G-P-P)₁₀ collagen-like domain (boldface), an extracellular domain (ECD) of human CD137L (from amino acid residue Ala93 to Glu254, UniProt accession #P41273), which is devoid of the coiled-coil trimerization motif (italics), followed by a TSMH affinity tag containing a thrombin cleavage site (LVPRGS), Strep-Tag II (WSHPQFEK), a c-myc epitope tag (EQKLISEEDL), and a polyhistidine tag (HHHHHH). The cDNA synthetic sequence (SEQ ID NO: 14) of the above polypeptide chain was codon-optimized by overlapping PCR and the PCR product was subcloned into a modified expression vector of pSecTag2/Hygro (Cat. No. V90020, Thermo Fisher Scientific Inc.), in which the original C-terminal affinity tag sequence was replaced with the TSMH affinity tag for detection and purification purposes.

SEQ ID NO: 13
METDTLLLWVLLLWVPGSTGDAAQPARRAKRSQPREPQVYTLPPSRDE
LTKNQVSLTCLVKGFYPSDIAVEWESNGQPENNYKTTPPVLDSDGSFF
LYSKLTVDKSRWQQGNVFSCSVMHEALHNHYTQKSLSLSPGAAAGSPG
PPGPPGPPGPPGPPGPPGPPGPPGPPGPPGPSSGGTGAQLVAQNVLLI
DGPLSWYSDPGLAGVSLTGGLSYKEDTKELVVAKAGVYYVFFQLELRR
VVAGEGSGSVSLALHLQPLRSAAGAAALALTVDLPPASSEARNSAFGF
QGRLLHLSAGQRLGVHLHTEARARHAWQLTQGATVLGLFRVTPEIPAG
LPSPRSEGSLVPRGSPGGSWSHPQFEKRRGGPEQKLISEEDLNSAVDG
SSHHHHHHSRGLE
SEQ ID NO: 14
ATGGAGACAGACACACTCCTGCTATGGGTACTGCTGCTCTGGGTTCCA
GGTTCCACTGGTGACGCGGCCCAGCCGGCCAGGCGCGCCAAACGATCA
CAGCCCCGAGAACCACAGGTGTACACCCTGCCCCCATCCCGGGATGAG
CTGACCAAGAACCAGGTCAGCCTGACCTGCCTGGTCAAAGGCTTCTAT
CCCAGCGACATCGCCGTGGAGTGGGAGAGCAATGGGCAGCCGGAGAAC
AACTACAAGACCACGCCTCCCGTGCTGGACTCCGACGGCTCCTTCTTC
CTCTACAGCAAGCTCACCGTGGACAAGAGCAGGTGGCAGCAGGGGAAC
GTCTTCTCATGCTCCGTGATGCATGAGGCTCTGCACAACCACTACACG
CAGAAGAGCCTCTCCCTGTCTCCGGGTGCGGCCGCTGGCAGCCCTGGG
CCACCTGGTCCCCCAGGTCCTCCAGGACCCCCAGGGCCCCCAGGCCCC
CCCGGGCCGCCTGGACCCCCAGGGCCACCAGGCCCCCCAGGCCCTTCC
TCTGGCGGAACCGGTGCCCAGCTGGTGGCTCAGAACGTCCTGCTGATC
GACGGACCACTGAGCTGGTACTCCGATCCCGGACTGGCTGGGGTGTCT
CTGACTGGCGGACTGAGTTATAAGGAGGATACCAAAGAACTGGTGGTC
GCAAAGGCCGGGGTGTACTATGTCTTCTTTCAGCTGGAGCTGAGGAGA
GTGGTCGCTGGGGAAGGTTCTGGCAGTGTGTCACTGGCACTGCACCTG
CAGCCACTGAGGTCTGCAGCTGGAGCAGCAGCTCTGGCACTGACAGTG
GACCTGCCACCTGCAAGCTCCGAGGCACGAAATTCCGCTTTCGGTTTT
CAGGGCAGGCTGCTGCACCTGAGCGCTGGACAGAGACTGGGGGTGCAC
CTGCATACCGAAGCTCGGGCACGCCATGCATGGCAGCTGACCCAGGGA
GCCACAGTCCTGGGGCTGTTTAGGGTCACTCCCGAGATTCCCGCCGGT
CTGCCTTCACCACGGTCTGAGGGATCCCTGGTGCCGCGCGGCAGCCCT
GGAGGCTCCTGGAGCCACCCGCAGTTCGAAAAGCGTCGAGGAGGGCCA
GAACAAAAACTCATCTCAGAAGAGGATCTGAATAGCGCCGTCGACGGC
AGCAGCCATCATCACCATCACCATAGCCGCGGACTCGAGTAA

### Example 3

### Expression, Purification, and Characterization of the Soluble Multimeric CD40 Ligand Fusion Proteins—PEP3CSCD40L, PEP3CS6CD40L, A20IDCS6CD40L, OVACS6CD40L, CD40LCS6OVA, CS6CD40L, and CS6hCD137L

The above expression plasmid constructs of PEP3CSCD40L, PEP3CS6CD40L, A20IDCS6CD40L, OVACS6CD40L, CD40LCS6OVA CS6CD40L, and CS6hCD137L were individually expressed in HEK293 cells using the Expi293 Expression System Kit (Cat. No. A14635, Thermo Fisher Scientific Inc.) according to the manufacturer's instructions. Expi293 cells were cultured in Expi293 expression media until reaching a density of 2.9×10⁶ cells/ml. These cultures were maintained on an orbital shaker (125 rpm) at 37° C. in an incubator with a humidified atmosphere containing 5% CO₂. For transfection, 30 μg of pure plasmid DNA and ExpiFectamine 293 Reagent were added separately to 1.5 ml aliquots of Opti-MEM Reduced Serum Medium at 37° C. After mixing, the solutions were incubated for 20 minutes at 25° C. before being added to the Expi293 cell culture. Cultures were then incubated in an orbital shaker (125 rpm) at 37° C. in an incubator with a humidified atmosphere containing 5% C02 for 48 to 72 hours. Afterwards, cells were harvested by centrifugation at 4000×g for 20 minutes at 4° C. For purification, approximately 30 ml each of the filtered culture medium was applied to a Strep-Tactin XT Superflow column (Cat. No. 2-1208-010, IBA Lifesciences GmbH) that had been pre-equilibrated with 1× buffer W (Cat. No. 2-1003-100, IBA Lifesciences GmbH) at a flow rate of 60 ml/h. Following washing with the same buffer, the proteins were eluted using buffer 1×BXT (Cat. No. 2-1042-025, IBA Lifesciences GmbH). SDS-PAGE was carried out by using a 4˜12% NuPAGE bis-Tris polyacrylamide gel with MES as running buffer (Cat. No. NP0002, Thermo Fisher Scientific Inc.). Proteins were stained with InstantBlue, (Cat. No. 516938 Expedeon, United States Biological). Bench Mark (Cat. No. 10747012, Thermo Fisher Scientific Inc.) were used as molecular size standard. Size exclusion (SEC)-HPLC analysis of the purified proteins were carried out by injecting 20 μg each of the purified PEP3CS6CD40L and PEP3CSCD40L proteins into a Zenix® SEC-300 Gel Filtration Column, 3 μm (Cat. No. Z777092, Merck KGaA) and absorbance at 280 nm were monitored.

Table 1 below summarizes the structural characters of the multi-trimeric fusion proteins after transient expression in Expi293 cells.

TABLE 1
Summary of multimeric fusion molecules used in this study

| | | Peptide | Extracellular | Predominate |
|---|---|---|---|---|
| Name | Format | name | Secretion | structure |
| PEP3CSCD40L | A | PEP3 | Yes | Trimer |
| PEP3CS6CD40L | B | PEP3 | Yes | Hexamer |
| A20IDCS6CD40L | B | A20 | Yes | Hexamer |
| OVACS6CD40L | B | OVA | Yes | Hexamer |
| CD40LCS6OVA | C | OVA | No | N/A¹ |
| CS6CD40L | D | N/A | Yes | Hexamer |
| CS6hCD137L | D | N/A | Yes | Hexamer |

¹Not applicable

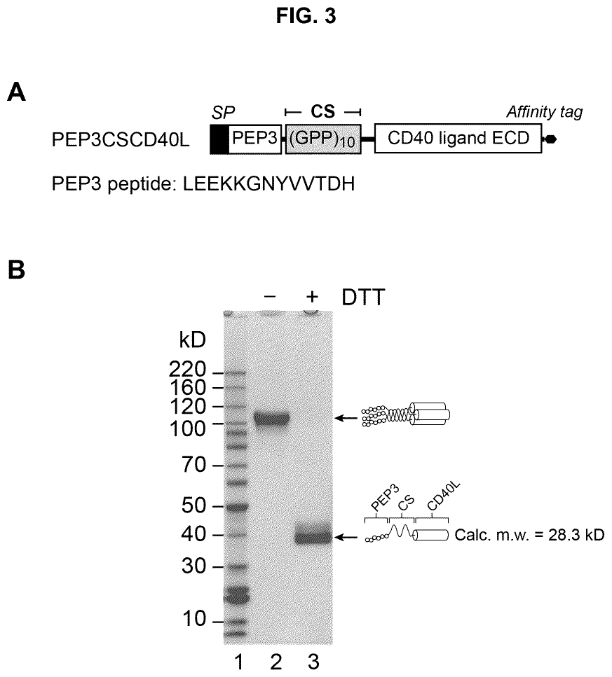

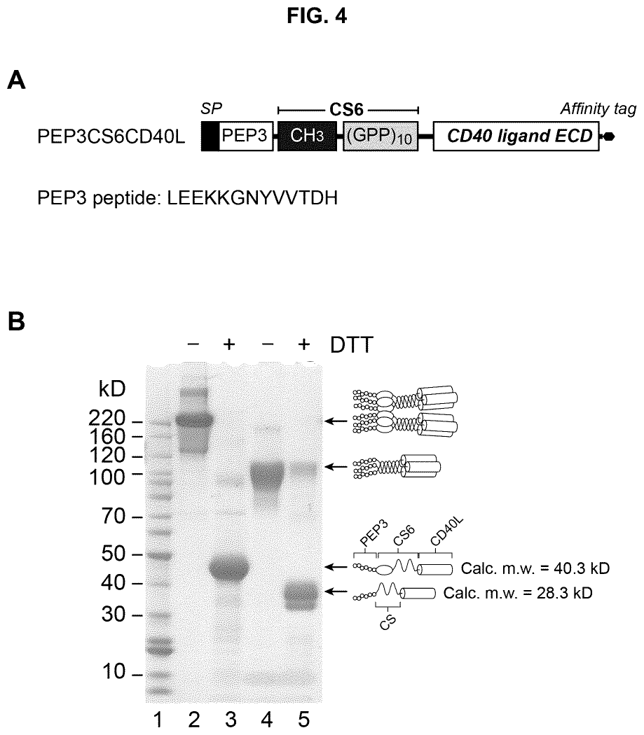

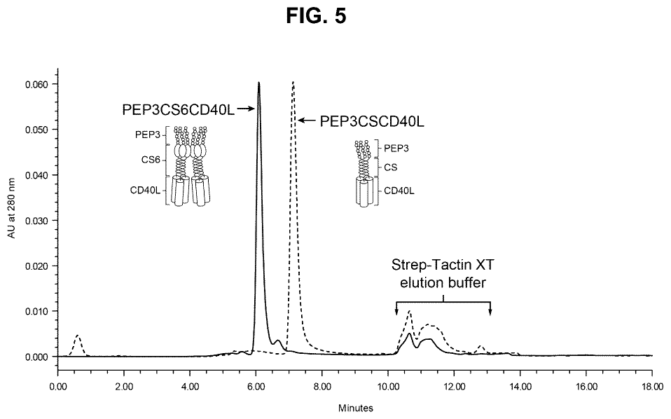

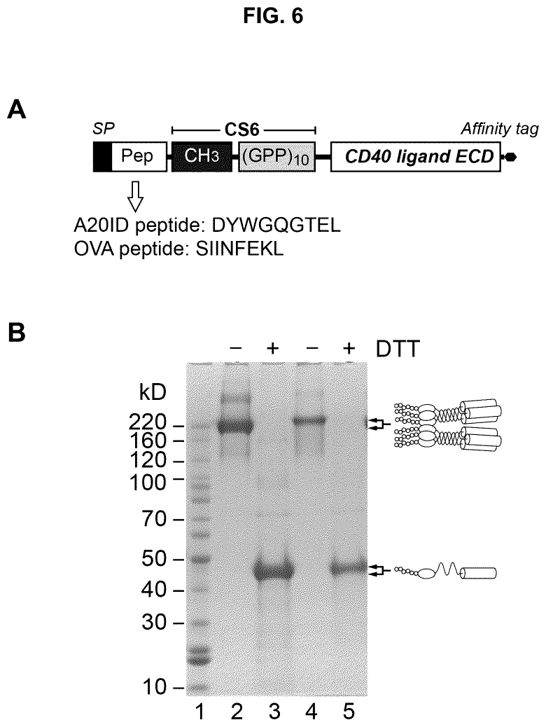

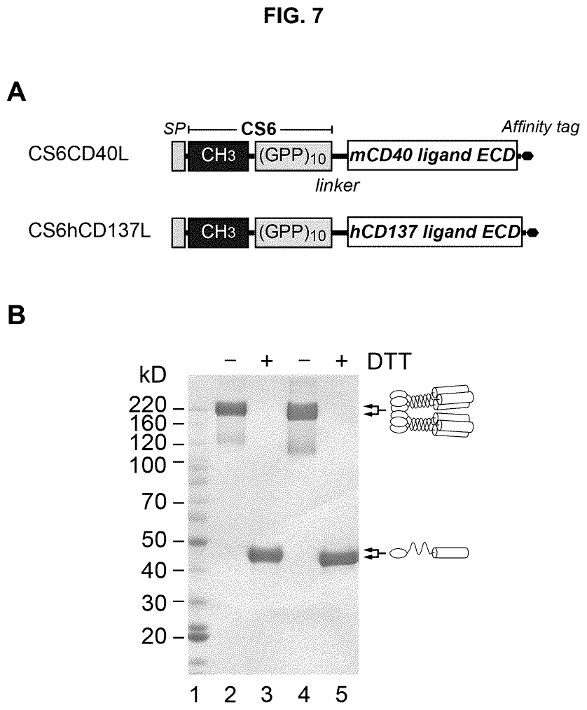

Both structural formats A and B, in which the ECD of CD40 ligand (devoid of the coiled-coil trimerization motif) is placed at the C-terminal of the CS and CS6 domains, respectively, can be expressed as soluble secretory proteins in Expi293 cells. See FIG. 2. SDS-PAGE and SEC-HPLC analyses of the Format A of PEP3CSCD40L under non-denaturation and phycological conditions, respectively, confirmed that the trimerizing motif of CS containing the collagen-like peptide, (GPP)₁₀ can form a stable CD40L trimer as shown in FIG. 3B, lane 2 and FIG. 4B, lane 4, and FIG. 5, dotted line. Under reducing conditions, PEP3CSCD40L trimer was dissociated into monomers (FIG. 3B, lane 3 and FIG. 4B, lane 5). SDS-PAGE and SEC-HPLC analyses of the Format B of PEP3CS6CD40L under non-denaturation and phycological conditions, respectively, the CS6 domain containing a human IgG₁ CH3 dimerizing domain and a trimerizing collagen-like peptide, (GPP)₁₀ can form a stable CD40L hexamer as shown in FIG. 4B, lane 2 and FIG. 5, solid line. Under reducing conditions, PEP3CSCD40L trimer was dissociated into monomers (FIGS. 4B, lane 3). Similar results were obtained in the Format B of A20IDCS6CD40L and OVACS6CD40L, which form stable hexamers analyzed by SDS-PAGE under non-denaturation conditions (FIG. 6). In structural format D, in which the N-terminal peptide motif is removed and the ECD of either mouse CD40L or human CD137L (devoid of the coiled-coil trimerization motif) is placed at the C-terminal of the CS6 domain, respectively (FIG. 7A), both of purified fusion proteins can form stable hexamers as analyzed by SDS-PAGE under non-denaturation conditions (FIG. 7B, lanes 2 and 4).

Unexpectedly, the structural Format C of CD40LCS6OVA in Table 1, where the ECD of CD40 ligand is placed at the N-terminal of the CS6 domain, followed by an OVA²⁵⁷⁻²⁶⁴ peptide, was found to be non-secretory in Expi293 cells. The TNF family of ligands are type II transmembrane proteins, in which the ECDs are located at the C-terminal region of the assembled protein trimer. Thus, correct domain structural orientation is required for proper CS6-CD40L fusion protein assembly into a hexamer.

### Example 4

### CD40 Reporter-Gene Potency Assay of the Soluble Multimeric CD40L Fusion Proteins

The potency of various CD40 agonist proteins, including PEP3CSCD40L, PEP3CS6CD40L, and anti-CD40 monoclonal antibody (clone G28.5), to activate cellular signaling was performed by a bioluminescent cell-based assay using a CD40 Bioassay Kit (Promega, #JA2151) according to the manufacturer's protocol. Briefly, frozen CD40 effector cells expressing CD40 and the downstream NF-kB promoter-driven firefly luciferase gene were thawed and seeded at a density of 2.5×10⁴ cells/well in 100 μl of recovery medium (90% RPMI 1640/10% FBS). Place lids on the assay plates and incubate in a 37° C., 5% CO2 incubator overnight (18-22 hours). On the day of assay, carefully remove all medium from cells in well. A series dilution of CD40 agonist proteins (70 μl in volume) was added into each well. Cells and CD40 agonist were cultured at 37° C. for 6 hours. Subsequently, 70 μl of One-Glo reagent were added and the plate was incubated at room temperature for 15 min. The reaction solution was then transferred into a white plate for luminescence measurement.

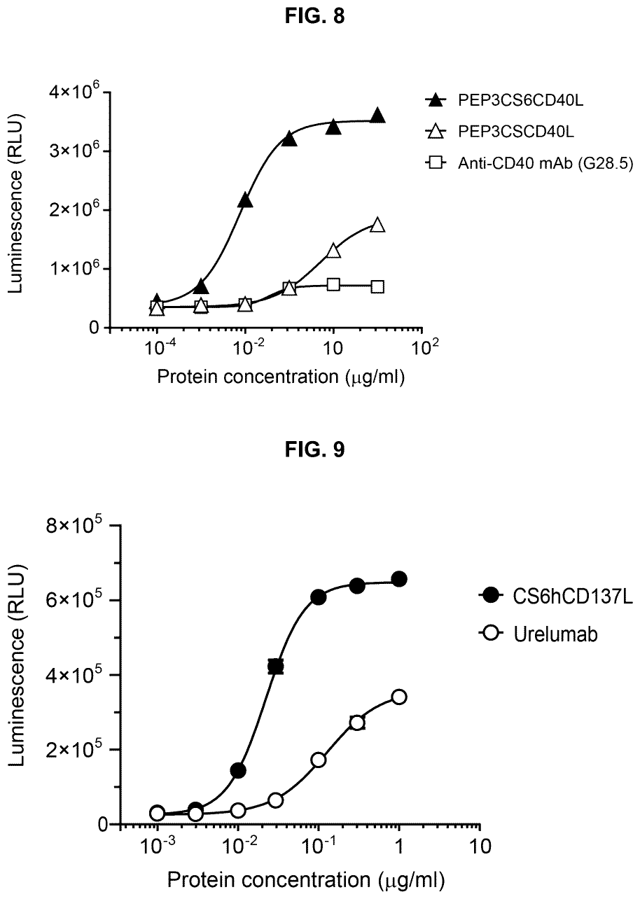

As shown in FIG. 8, upon binding to the CD40 of the reporter cells, the soluble PEP3CS6CD40L hexamer exhibits the strongest downstream expression of firefly luciferase than the soluble PEP3CSCD40L trimer and the bivalent CD40 agonist monoclonal antibody (G28.5). The results also confirm that the structures of the hexameric PEP3CS6CD40L and the trimeric PEP3CSCD40L fusion proteins are functionally folded, capable of eliciting a strong downstream cellular signaling effect for CD40-response of cell activation.

### Example 5

### CD137 Reporter-Gene Potency Assay of the Soluble Hexameric CD137L Fusion Protein

Urelumab is an anti-human CD137 agonist monoclonal antibody whose purpose is to stimulate T cells for promoting anti-tumor immunity. The potency of the soluble hexameric CD137L fusion protein, CS6hCD137L, and the CD137 agonist monoclonal antibody (Urelumab analog), to activate cellular signaling was performed by a bioluminescent cell-based assay using a CD137 Bioassay Kit (Promega, #JA2351) according to the manufacturer's protocol. Briefly, frozen CD137 effector cells expressing CD137 and the downstream NF-kB promoter-driven firefly luciferase gene were thawed and seeded at a density of 2.5×10⁴ cells/well in 100 μl of recovery medium (95% RPMI 1640/5% FBS). Place lids on the assay plates and incubate in a 37° C., 5% CO2 incubator overnight (18-22 hours). On the day of assay, carefully remove all medium from cells in well. A series dilution of CD40 agonist proteins (70 μl in volume) was added into each well. Cells and CD40 agonist were cultured at 37° C. for 6 hours. Subsequently, 70 μl of One-Glo reagent were added and the plate was incubated at room temperature for 15 min. The reaction solution was then transferred into a white plate for luminescence measurement.

As shown in FIG. 9, upon binding to the CD137 of the reporter cells, the soluble CS6hCD137L hexamer exhibits a stronger downstream expression of firefly luciferase than the bivalent CD137 agonist monoclonal antibody (Urelumab analog). The results also confirm that the structures of the hexameric CS6hCD137L is functionally folded, capable of eliciting a strong downstream cellular signaling effect for CD137-response of cell activation through CD137-CD137L clustering.

### Example 6

### Dendritic Cell Activation by the Soluble Multimeric CD40L Fusion Proteins

Comparison of various CD40L fusion proteins in the activation of CD40⁺ dendritic cells was performed. MutuDC1940 dendritic cells (Cat. No. T0528, Applied Biological Materials Inc.) ¹³ were plated at 5×10⁵ cells/well in a 12-well plate and incubated at 37° C. in RPMI-1640 medium supplemented with L-glutamine, 50 μM β-mercaptoethanol, 10 μg/ml gentamycin and 10% heat-inactivated fetal bovine serum in the presence of 1 μg/ml of recombinant mouse CD40L (active trimer, Cat. No. CDL-M52D5, Acro Biosystems), PEP3CSCD40L and PEP3CS6CD40L for 24 h. Cells were washed, re-suspended in staining buffer and stained with PE-conjugated anti-mouse MHC class II mAb, PerCP/Cy5.5-conjugated anti-mouse CD40 and APC conjugated anti-mouse CD86 before subject to flow cytometric analysis.

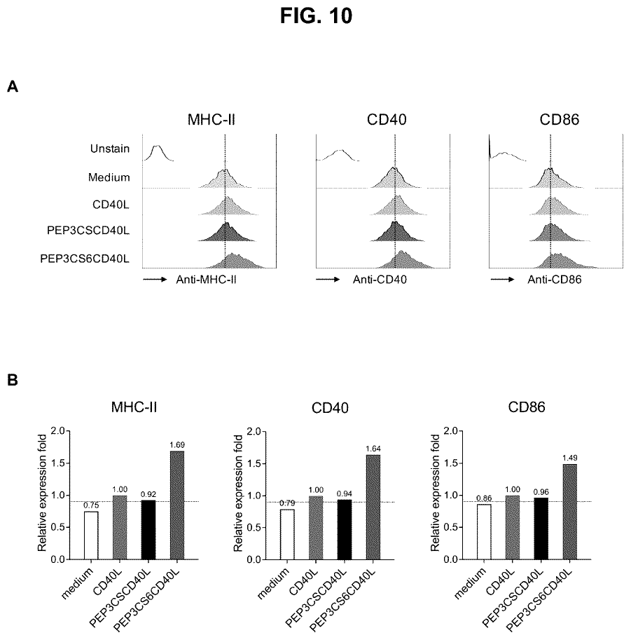

As shown in FIG. 10A, flow cytometry data analysis showed that only the soluble PEP3CS6CD40L hexamer, but not the recombinant mouse CD40L (active trimer) or PEP3CSCD40L trimer, can significantly up-regulated the expression of dendritic cell activation surface markers of MHC class II, CD40, and CD86, of which the induction levels for PEP3CS6CD40L are around 1.5-fold higher than the recombinant mouse CD40L reference control (FIG. 10B).

The results demonstrate that multivalent crosslinking of the CD40L with CD40 receptors on dendritic cells is essential for dendritic cell activation and maturation. The results further demonstrate that hexameric CD40L fusion protein (PEP3CS6CD40L) has a stronger stimulating activity of MutuDC1940 dendritic cells than either the native soluble trimeric CD40L or the recombinant PEP3CSCD40L trimer.

### Example 7

Exogenous Antigen Presentation by Dendritic Cells Treated with OVA257-264 Antigen Peptide and OVACS6CD40L Hexamer Protein

MutuDC1940 dendritic cells were plated at 5×10⁵ cells/well in a 12-well plate in the presence of different concentrations of OVA²⁵⁷⁻²⁶⁴ peptide and OVACS6CD40L hexamer protein for 24 h. Cells were washed, re-suspended in staining buffer and stained with an APC-conjugated 25D1.16 monoclonal antibody (Cat. No. 141605, Biolegend), which binds to OVA peptide²⁵⁷⁻²⁶⁴ and H-2K^{b} of MHC class I complex specifically, before subject to flow cytometric analysis.

In FIG. 11, the 25D1.16 monoclonal antibody specifically reacted with OVA²⁵⁷⁻²⁶⁴ peptide bound to H-2Kb of MHC class I, indicating that the OVA²⁵⁷⁻²⁶⁴ peptide successfully loaded on the H-2K^{b} of MHC class I of MutuDC1940 cells at a saturate peptide concentration of around 2 μg/ml. No binding was found in MutuDC1940 cells treated with OVACS6CD40L. Even though the soluble hexameric CD40L fusion protein of OVACS6CD40L can activate MutuDC1940 cells, the OVA²⁵⁷⁻²⁶⁴ peptide in OVACS6CD40L cannot be processed by the dendritic cells exogenously and loaded on the H-2K^{b} of MHC class I of the dendritic cell surface. The results confirm that class I MHC molecules utilize the cytosolic pathway for processing antigens.

### Example 8

In Vitro Synthesis of 5′-Capped, Pseudouridine-Modified mRNAs Coding for the Soluble Multimeric CD40L Fusion Proteins

In order to have a dual function of simultaneously activation of the same dendritic cell by the intact secretory hexameric OVACS6CD40L protein and the OVA²⁵⁷⁻²⁶⁴ antigen peptide processing and presentation in OVACS6CD40L to class I MHC molecules, we sought to express the protein endogenously either by introducing a DNA coding for OVACS6CD40L or by directly delivery of an mRNA having the open reading frame of OVACS6CD40L to dendric cells directly. The cDNA open reading frame sequences coding for the soluble multimeric CD40L fusion proteins, including PEP3CSCD40L, PEP3CS6CD40L, OVACS6CD40L, and CS6CD40L (devoid of the N-terminal antigen peptide sequence), were subcloned in-frame into the multiple cloning site (MCS) of a pUC-T7ag mRNA expression vector. The pUC-T7ag vector contains a di-nucleotide “AG” immediately downstream of a T7 promoter for CleanCap® AG 5′-end capping, followed by a 5′UTR, MCS, a TGA translation stop codon, 3′ UTR, and a poly(A) tail. In vitro transcription of various soluble multimeric CD40L mRNAs was performed by using the RiboMAX™ Large Scale RNA Production System (Cat. No. P1300, Promega, Corp.). Each transcription reaction contained 10 μg of linearized pUC-T7ag DNA templates, 1× reaction buffer, 4 mM of CleanCap® AG (Cat. No. N-7113, TriLink BioTechnologies), 5 mM each of ATP, GTP, CTP, N1-methylpseudo-UTP (Cat. No. NU-890L, Jena bioscience), 100 units of RNasin® Plus ribonuclease inhibitor (Cat. No. N2615, Promega, Corp.), and 100 units of T7 RNA polymerase at a final volume of 100 μl. The reaction mixture was incubated at 37° C. for 3 hours, followed by 10 units of DNase I (Cat. No. M6101, Promega Corp.) digestion for 15 minutes at 37° C. to remove the DNA template. The mRNAs were purified by an Embark™ mRNA Purification Kit (Cat. No. EMBARK50, Messenger Bio) following the manufacturer's instruction. The amount of RNA was determined by UV absorbance at 260 nm.

### Example 9

mRNA Transfection and Western Blot Analysis

Since the effectiveness of mRNA therapeutics depends on their protein translational level and duration after delivering of the mRNA into the designated cells, the mRNA translational level and duration of the soluble multimeric CD40L fusion proteins, including PEP3CSCD40L and PEP3CS6CD40L were evaluated. HEK293T cells were transfected with 1 μg each of the in vitro transcribed PEP3CSCD40L and PEP3CS6CD40L mRNAs in a 24-well plate using Lipofectamine MessengerMAX (Cat. No. LMRNA015, Thermo Fisher Scientific Inc.) according to the manufacturer's protocols. Cells were cultured at 37° C. in Dulbecco's modified Eagle's Medium (DMEM) medium (Invitrogen) supplemented with 10% fetal bovine serum (FBS). Thirty microliters of the culture supernatants were collected on day 1, 2 and 3 post-transfection and were run on a 4˜12% NuPAGE bis-Tris SDS-polyacrylamide gel with MES as running buffer (Cat. No. NP0002, Thermo Fisher Scientific Inc.) under non-denaturing conditions. The proteins were then transferred onto a nitrocellulose membrane (GE Healthcare) according to the manufacturer's protocol. Following blocking with 5% skim milk overnight at 4° C., the membrane was incubated with an EGFRvIII-specific rabbit monoclonal antibody, clone RM419 (Cat. No. 31-1305-00, RevMAb Biosciences) overnight at 4° C. After washing, the membrane was incubated with a horseradish peroxidase (HRP)-linked anti-rabbit IgG antibody (Cat. No. 7074S, Cell Signaling,) at room temperature for 1 hour. The membrane was treated with ECL Western Blotting Detection Reagent (Invitrogen) and was imaged using a Fujifilm LAS-4000 detection system.

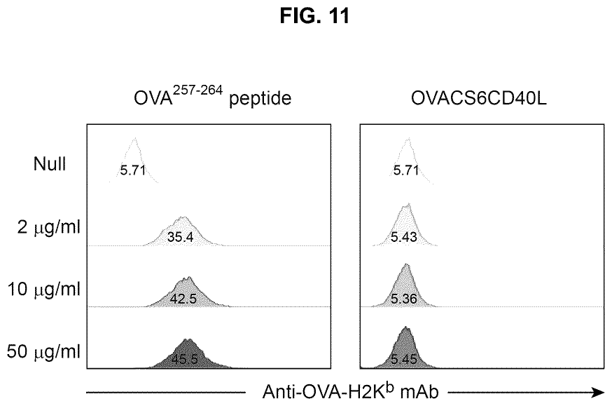

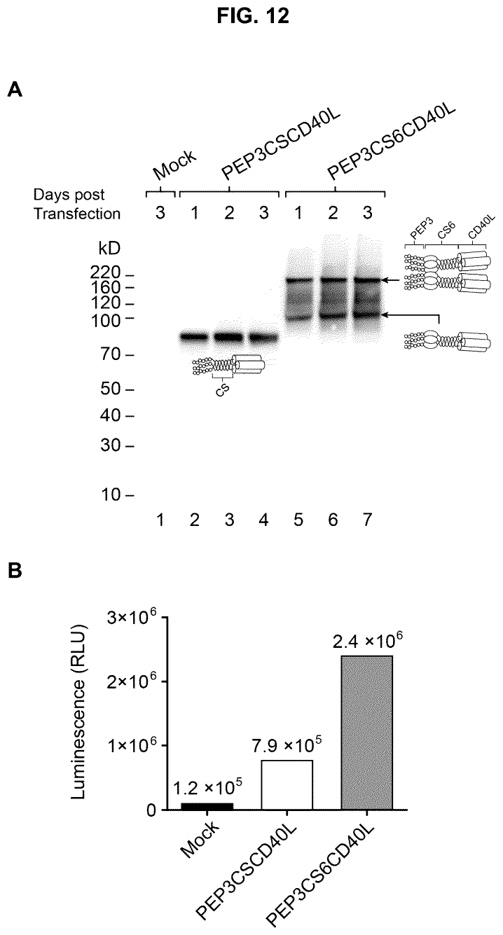

FIG. 12A shows the immunoblot image results. As expected from the recombinant expression of both PEP3CSCD40L and PEP3CS6CD40L proteins by transient transfection of their DNA plasmid constructs into Expi293 cells, direct delivery of the mRNA coding for the two proteins to HEK293T cells can successfully secrete intact trimeric and hexameric PEP3 antigen peptide-containing CD40L fusions, respectively, after probing with the EGFRvIII-specific antibody. Notably, a trimeric form with half the molecular size of PEP3CS6CD40L hexamer was detected by the antibody. This trimer species is likely caused by SDS dissociation of the non-interchain disulfide bonded CH3 dimer under electrophoresis. Comparison of the protein expression level from the 1-μg mRNA-transfected HEK293T cells over the time course indicated that the mRNA-transfected cells can constitutively express both proteins for up to 3 days.

### Example 10

CD40 Reporter-Gene Potency Assay of the mRNA-Transfected Cell Culture Supernatants

To further evaluate the CD40 activation potency of the above cell secretory multimeric CD40L fusion proteins synthesized by their cognate mRNAs, including PEP3CSCD40L and PEP3CS6CD40L, a cell-based, luciferase reporter gene assay using a CD40 Bioassay Kit (Promega, #JA2151) to activate cellular signaling was performed as described in EXAMPLE 4. Forty microliters of the mRNA-transfected cell culture supernatants of PEP3CSCD40L and PEP3CS6CD40L on 3-day post-transfection were added into each well. Cells were cultured at 37° C. for 6 hours. Subsequently, 70 μl of One-Glo reagent were added and the plate was incubated at room temperature for 15 min. The reaction solution was then transferred into a white plate for luminescence measurement.

FIG. 12B shows that the culture supernatants from the PEP3CSCD40L and PEP3CS6CD40L mRNA-transfected HEK293T cells can activate CD40-bearing reporter cells. In similar to the results of EXAMPLE 4 by using the purified proteins, the mRNA coding for the hexameric PEP3CS6CD40L exerts a 3-fold CD40 activating potency than that of the trimeric PEP3CSCD40L.

### Example 11

Dendritic Cell Activation by the Cell-Transfected mRNA Coding for the Soluble Multimeric CD40L Fusion Proteins

CD40⁺ MutuDC1940 dendritic cells (Cat. No. T0528, Applied Biological Materials Inc.) were plated at 5×10⁵ cells/well in a 12-well plate and incubated at 37° C. in RPMI-1640 medium supplemented with L-glutamine, 50 μM β-mercaptoethanol, 10 μg/ml gentamycin and 10% heat-inactivated fetal bovine serum. Cells were transfected with either 1 or 2 μg of the in vitro transcribed PEP3CS6CD40L mRNAs in a 24-well plate using Lipofectamine MessengerMAX (Cat. No. LMRNA015, Thermo Fisher Scientific Inc.) according to the manufacturer's protocols. After 24 hours of transfection, cells were washed, re-suspended in staining buffer, and stained with PE-conjugated anti-mouse MHC class II mAb, PerCP/Cy5.5-conjugated anti-mouse CD40 and APC conjugated anti-mouse CD86 before subject to flow cytometric analysis.

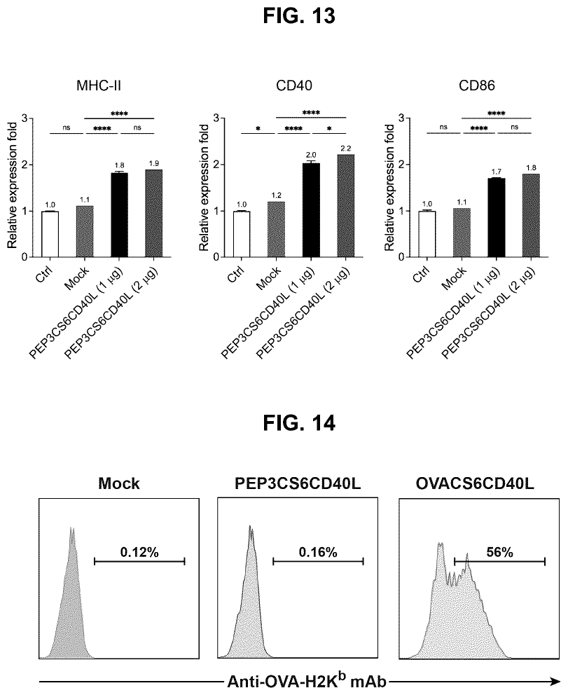

As shown in FIG. 13, MutuDC1940 dendritic cells can be activated after transfection with the mRNA coding for the hexameric CD40L fusion protein (PEP3CS6CD40L). Flow cytometry data analysis showed that MutuDC1940 dendritic cells transfected with either 1 or 2 μg of mRNA coding for PEP3CS6CD40L hexamer can significantly up-regulate the expression of dendritic cell activation surface markers of MHC class II, CD40, and CD86, of which the induction levels for PEP3CS6CD40L are around 1.8-, 2.0-, 1.7-fold higher than the non-transfected cell control (Ctrl), respectively. The results indicate that the mRNA coding for PEP3CS6CD40L hexamer can be successfully secreted as a soluble protein, which can bind and activate the CD40+ dendritic cells.

### Example 12

Endogenous Antigen Presentation by Dendritic Cells Transfected with the mRNAs Coding for OVACS6CD40L

MutuDC1940 dendritic cells were plated at 5×10⁵ cells/well in a 12-well plate the day before transfection and incubated at 37° C. in RPMI-1640 medium supplemented with L-glutamine, 50 μM β-mercaptoethanol, 10 μg/ml gentamycin and 10% heat-inactivated fetal bovine serum. Cells were transfected with 1p g each of PEP3CS6CD40L mRNA or OVACS6CD40L mRNA using Lipofectamine MessengerMAX for 24 hours. Cells were washed, re-suspended in staining buffer and stained with an APC-conjugated 25D1.16 monoclonal antibody (Cat. No. 141605, Biolegend), which binds to OVA peptide²⁵⁷⁻²⁶⁴ and H-2K^{b} of MHC class I complex specifically, before subject to flow cytometric analysis.

In FIG. 14, the 25D1.16 monoclonal antibody specifically reacts with OVACS6CD40L mRNA-transfected MutuDC1940 dendritic cells, indicating that OVA²⁵⁷⁻²⁶⁴ peptide successfully loaded and formation of the H-2K^{b} of MHC class I-peptide complex. The results demonstrated that, through an endogenously antigen presentation pathway of delivering OVACS6CD40L mRNA into MutuDC1940 dendritic cells, the OVA antigenic peptide can be processed and transferred onto MHC class I complex at the cell surface. No binding was found in MutuDC1940 cells treated with mock (transfection reagent only) or PEP3CS6CD40L mRNA, confirming the antibody specificity of 25D1.16.

The previous results in EXAMPLE 7 shows that by treating MutuDC1940 cells with intact OVACS6CD40L protein, the OVA peptide²⁵⁷⁻²⁶⁴ antigen peptide cannot be processed and loaded on class I MHC molecules on cell surface. However, through an endogenous protein expression pathway (i.e. protein generated within the cell) by delivery of an mRNA having the open reading frame of OVACS6CD40L to dendric cells directly, the OVA²⁵⁷⁻²⁶⁴ peptide can be successfully processed and loaded on class I MHC molecules on cell surface.

### Example 13

### Antigen Presentation and CD8+ T Cell Activation by Dendritic Cells

To further demonstrate that the amount of OVA²⁵⁷⁻²⁶⁴ peptide/MHC class I complexes on MutuDC1940 cells in EXAMPLE 12 can prime and activate an OVA²⁵⁷⁻²⁶⁴ peptide-specific CD8⁺ T cells, the OVACS6CD40L mRNA-transfected MutuDC1940 cells were co-cultured with the CD8⁺ T cells to see if the T cells can be activated through IL-2 secretion. MutuDC1940 dendritic cells were plated at 5×10⁵ cells/well in a 12-well plate the day before transfection and incubated at 37° C. in RPMI-1640 medium supplemented with L-glutamine, 50 μM β-mercaptoethanol, 10 μg/ml gentamycin and 10% heat-inactivated fetal bovine serum. MutuDC1940 cells were transfected with 1 μg of CS6CD40L mRNA or OVACS6CD40L mRNA using Lipofectamine MessengerMAX or stimulated with 2 μg/ml of OVA²⁵⁷⁻²⁶⁴ peptide or 10 μg/ml of OVACS6CD40L. After 24 h incubation, OVA²⁵⁷⁻²⁶⁴ peptide-specific CD8+ T cell hybridoma RF33.70 cells (kindly provided by Kenneth Rock, University of Massachusetts) were added into the 12-well plate at a density of 1×10⁶ cells/well and co-cultured with MutuDC1940 cells for another 48 h. The supernatants from these cultures were analyzed for the presence of IL-2 using HEK-Blue™ IL-2 cells (InvivoGen) according to the manufacturer's protocols.

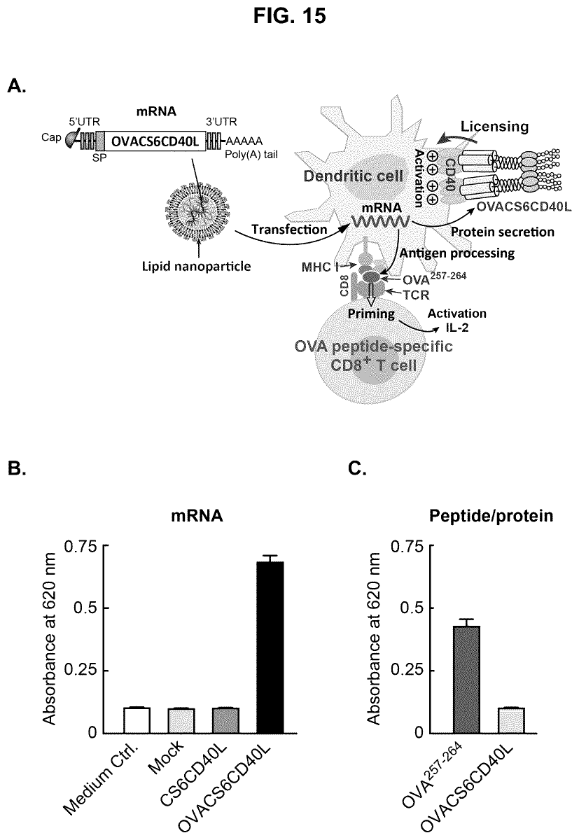

The schematic representation of direct T-cell priming and activation with OVACS6CD40L mRNA-transfected dendritic cells is illustrated in FIG. 15A. The OVACS6CD40L mRNA-transfected MutuDC1940 dendritic cells can prime and activate the OVA antigen peptide-specific CD8+ T cells. The mRNA-transfected dendritic cell produces secretory hexameric OVACS6CD40L protein and self-activated through binding to its own CD40 receptor. In the meantime, part of the cytosolic OVACS6CD40L protein is degraded through proteasome system, followed by presenting the OVA²⁵⁷⁻²⁶⁴ antigen peptide to class I MHC molecule. The OVA²⁵⁷⁻²⁶⁴-MHC class I complex of the licensed dendritic cells can react (prime) with the OVA²⁵⁷⁻²⁶⁴ antigen peptide-specific CD8+ T cells, leading to T-cell activation and expansion by secretion of cytokine IL-2.

Only the OVACS6CD40L mRNA, but not the mock or non-OVA antigen peptide bearing CS6CD40L mRNA, after transfecting MutuDC1940 cells, can prime and activate RF33.70 cells (FIG. 15B). As expected in results of EXAMPLE 7, exogenously treatment of the OVA²⁵⁷⁻²⁶⁴ peptide, the peptide specific CD8⁺ T cells could be activated by the engagement of TCR with the loaded OVA²⁵⁷⁻²⁶⁴ peptide/MHC class I complexes on MutuDC1940 cells, but not to the unprocessable OVACS6CD40L protein (FIG. 15C).

### Example 14

Anti-Tumor Efficacy of PEP3CS6CD40L mRNA-LNP in a Syngeneic Mouse Glioblastoma Multiforme (GBM) Tumor Model

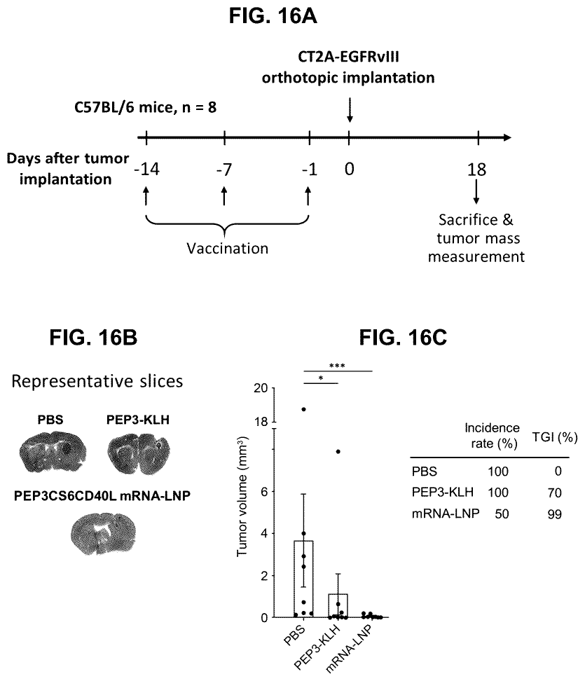

For syngeneic mouse GBM tumor model experiments, 6- to 8-week-old female C57BL/6 mice were randomly divided into three groups (n=8 for each group) and were dosed as depicted in FIG. 16A. In group 1, each mouse received 100 μl of PBS (control) by i.v. injection on days −14, −7, and −1. In group 2, each mouse received 20 μg of PEP3CS6CD40L mRNA formulated with in vivo-jetRNA®+(Polyplus, Cat. #101000122) lipid nanoparticles by i.v. injection on days −14, −7, and −1. In group 3, each mouse received 50 μg of PEP3-KLH protein mixed with complete Freund's adjuvant on day −14, followed by another 50 μg of PEP3-KLH protein mixed with incomplete Freund's adjuvant on day −1 by s.c. injection. After the last administration, mice were intracranially implanted with 1×10⁵ cells of mouse CT-2A glioblastoma multiforme (GBM) cells (EMD Millipore Corporation, Cat. #SCC194) stably expressing EGFRvIII gene (CT-2A-EGFRvIII) on day 0. Mice were sacrificed on day 18 and the tumor volume was calculated by the sum of the tumor area in each slice multiplied by the thickness and gap per slice (FIG. 16B, representative slices).

The results shown in FIGS. 16B and 16C indicate that after immunization, both PEP3CS6CD40L mRNA-LNP and PEP3-KLH/adjuvant vaccines can effectively prevent the growth of CT-2A-EGFRvIII GBM in C57BL/6 mice as compared with the PBS non-treated group. Notably, the anti-tumor effect of PEP3CS6CD40L mRNA-LNP vaccine is far superior to the PEP3-LKH/adjuvant vaccine in terms of tumor size and tumor incidence rate, in which 4 in 8 mice were found to be tumor-free in the PEP3CS6CD40L mRNA-LNP vaccinated group.

### Example 15

Anti-Tumor Efficacy of OVACS6CD40L mRNA-LNP in a Syngeneic Lymphoma Tumor Model

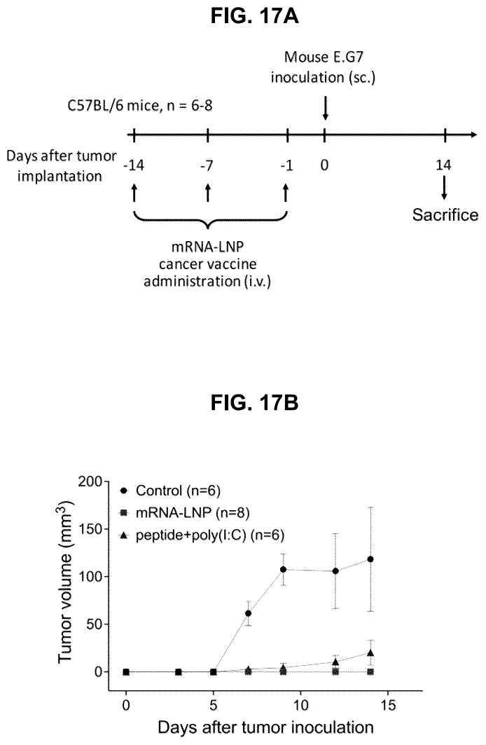

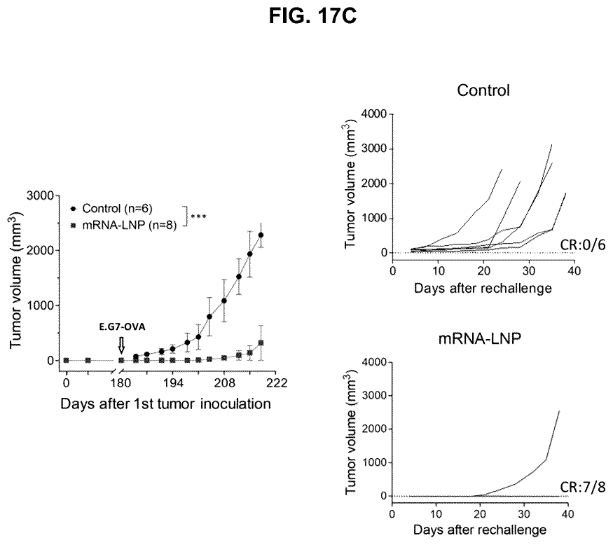

For syngeneic mouse lymphoma tumor model experiments, 6- to 8-week-old female C57BL/6 mice were randomly divided to three groups and were dosed as depicted in FIG. 17A. In group 1 (n=6), each mouse received 100 μl of PBS (control) by i.v. injection. In group 2 (n=8), each mouse received 20 μg of OVACS6CD40L mRNA formulated with in vivo-jetRNA®+(Polyplus, Cat. #101000122) lipid nanoparticles by i.v. injection. In group 3 (n=6), each mouse received 100 μg of OVA²⁵⁷⁻²⁶⁴ peptide mixed with 50 μg of poly (I:C) adjuvant (cat no. vac-pic, InvivoGen) by i.p injection. Mice in groups 1 and 2 were repeatedly administered on days −14, −7, and −1, whereas mice in group 3 were administered on days −14, and −1, before subcutaneously implanted with 3×10⁵ of E.G7-OVA cells (cat no. 60418, Bioresource Collection and Research Center, Taiwan) on the lower flank region of mice on day 0. Mice were sacrificed on day 14. To monitor tumor progression, tumor sizes were measured continually and represented as ½×L×W² (cubic millimeters), where L is the longest and W is the shortest tumor diameter.

Tumor rechallenge studies were designed as follows: Tumor-free mice in the OVACS6CD40L mRNA-LNP treated group were monitored for 180 days to confirm that there was no sign of tumor growth. Afterward, the mice (n=8) were subcutaneously implanted with 3×10⁵ of E.G7-OVA cells and tumor growth was monitored individually by measuring the tumor size once per week for 35 days. Six 6- to 8-week-old female C57BL/6 naïve mice were implanted with the same amount of tumor cells as the control group.

The results shown in FIG. 17B indicate that the anti-tumor effect of OVACS6CD40L mRNA-LNP vaccine is far superior to the OVA peptide/adjuvant vaccine. All eight mice that pre-treated with 3 doses of OVACS6CD40L mRNA-LNP showed complete tumor rejection for up to 180 days after E.G7-OVA cell inoculation. Two in 6 mice, in the OVA peptide/poly (I:C) adjuvant group, were found to be positive in tumor growth on day 14. In the PBS control group, 4 in 6 mice, were found to be positive in tumor growth on day 14.

In FIG. 17C, the above eight OVACS6CD40L mRNA-LNP treated mice that had immunity to E.G7-OVA cells were rechallenged with E.G7-OVA cells. After 35 days of monitoring tumor growth, only 1 in 8 mice was found to have tumor growth. In contrast, all six naïve mice receiving E.G7-OVA cells were found to be positive in tumor growth and tumor volume increases exponentially over time.

These results demonstrate that OVACS6CD40L mRNA-LNP can not only prevent the growth of syngeneic E.G7-OVA lymphoma cells in C57BL/6 mice, but it exerts a long-term cytotoxic T memory effect, presumably caused by the activation of CD40 on dendritic cells through clustering of the secretory form of hexameric CD40L fusion coded by OVACS6CD40L mRNA.

Without further elaboration, it is believed that one skilled in the art can, based on the description herein, utilize the present invention to its fullest extent. All publications cited herein are hereby incorporated by reference in their entirety.

1. A method for producing a multimeric fusion protein, the method comprising:
expressing in a mammalian cell a nucleic acid coding for an amino acid sequence comprising, in an N-terminal to C-terminal direction, a signal peptide, optionally an antigenic peptide, a CH3 domain of human IgG₁, a (G-P-P)₁₀ collagen-like domain, and a TNF ligand superfamily extracellular domain, the extracellular domain being devoid of a coiled-coil trimerization motif; and
allowing the polypeptides expressed in the mammalian cell from the nucleic acid to at least one of trimerize and hexamerize into one or more multimeric fusion proteins.

2. The method according to claim 1, wherein the mammalian cell is an antigen presenting cell.

3. The method according to claim 1, wherein the antigenic peptide comprises one selected from the group consisting of epidermal growth factor receptor variant III peptide (PEP3), chicken ovalbumin (257-264) antigen peptide (OVA), and idiotypic antibody peptide derived from a BALB/c B cell lymphoma line A20 (A20ID).

4. The method according to claim 1, wherein the TNF ligand superfamily extracellular domain comprises CD40L.

5. The method according to claim 1, wherein the antigenic peptide forms a peptide-major histocompatibility complex (MHC) protein complex on surfaces of an antigen presenting cell.

6. The method according to claim 5, wherein the antigenic peptide-MHC protein complex is configured to engage T cell receptors on antigen peptide-specific T cells to stimulate an immune response.

7. The method according to claim 1, wherein the TNF ligand superfamily extracellular domain comprises CD137L.

8. The method according to claim 2, wherein the antigen presenting cell comprises a dendritic cell.

9. The method according to claim 1, wherein a nucleotide sequence of the nucleic acid is SEQ ID NO: 4, SEQ ID NO: 6, or SEQ ID NO: 8, or a sequence having at least 90% sequence identity to the nucleotide sequence.

10. A method for producing a multimeric fusion protein, the method comprising expressing in an antigen presenting cell a nucleic acid coding for an amino acid sequence set forth in SEQ ID NO: 3, SEQ ID NO: 5, or SEQ ID NO: 7, or an amino acid sequence having at least 90% sequence identity to the amino acid sequence set forth in SEQ ID NO: 3, SEQ ID NO: 5, or SEQ ID NO: 7.

11. A soluble multimeric fusion protein comprising, from its N-terminus to C-terminus: optionally an antigenic peptide, a CH3 domain of human IgG₁, a (G-P-X1)₁₀ collagen-like domain, wherein X1 comprises P or O, and a TNF ligand superfamily extracellular domain, the extracellular domain being devoid of a coiled-coil trimerization motif, wherein the soluble multimeric fusion protein has a hexameric structure.

12. An isolated nucleic acid encoding the soluble multimeric fusion protein of claim 11.

13. An isolated expression vector comprising the nucleic acid of claim 12.

14. An isolated antigen presenting cell comprising the expression vector of claim 13.

15. An isolated nucleic acid consisting of a nucleic acid coding for an amino acid sequence set forth in SEQ ID NO: 3, SEQ ID NO: 5, or SEQ ID NO: 7, or an amino acid sequence having at least 90% sequence identity to the amino acid sequence set forth in SEQ ID NO: 3, SEQ ID NO: 5, or SEQ ID NO: 7.

16. A pharmaceutical composition comprising a nucleic acid coding for a soluble multimeric fusion protein of claim 11.

17. The pharmaceutical composition of claim 16, further comprising a pharmaceutically acceptable carrier, excipient, or diluent.

18. The pharmaceutical composition of claim 16 for use in treatment of cancer.

19. A method for treatment of cancer, the method comprising administering to a subject in need thereof the pharmaceutical composition of claim 16.

20. The method according to claim 19, wherein the administering comprises delivering the pharmaceutical composition to dendritic cells of the subject ex vivo via transfection.

21. The method according to claim 19, wherein the administering comprises delivering the pharmaceutical composition to dendritic cells of the subject in vivo via a lipid nanoparticle.

22. The method according to claim 19, wherein the cancer is at least one selected from the group consisting of lymphoma, breast cancer, lung cancer, colon cancer, rectal cancer, prostate cancer, melanoma, brain cancer, spinal cord cancer, ovarian cancer, pancreatic cancer, uterine cancer, and kidney cancer.

23. A method for controlling an immune response, the method comprising delivering the pharmaceutical composition of claim 16 to dendritic cells of a lymphatic system.

24. The method according to claim 23, wherein the pharmaceutical composition activates CD40 on the dendritic cells and induces CD8+ T cells into memory cells through MHC class I antigenic peptide presentation.
